(19) United States
(12) Patent Application Publication
(10) Pub. No.: US 2025/0361493 A1
(43) Pub. Date: Nov. 27, 2025

(54) MUTANT NUDIVIRUS INSECT CONTROL

(71) Applicant: University of Kentucky Research Foundation, Lexington, KY (US)

(72) Inventors: Bruce WEBB, Lexington, KY (US); Mary WALLACE, Lexington, KY (US); Jimmy MAINS, Morehead, KY (US); Michael ANDERSON, Berea, KY (US); Megha KALSI, Plymouth, MA (US)

(21) Appl. No.: 19/232,773

(22) Filed: Jun. 9, 2025

Related U.S. Application Data
(63) Continuation-in-part of application No. 18/066,224, filed on Dec. 14, 2022.
Application No. 18/066,224 is a continuation-in-part of application No. 16/722,071, filed on Dec. 20, 2019, now Pat. No. 11,591,575.
Application No. 16/722,071 is a continuation of application No. 15/694,323, filed on Sep. 1, 2017, now abandoned.
Application No. 15/694,323 is a continuation of application No. 15/154,069, filed on May 13, 2016, now Pat. No. 9,770,033.
(60) Provisional application No. 62/161,674, filed on May 14, 2015.

(51) Int. Cl. C12N 7/00; A01N 63/40

(52) U.S. Cl. CPC C12N 7/00; A01N 63/40; C12N 2710/00021; C12N 2710/00031; C12N 2710/00071

(57) ABSTRACT

Disclosed are the H. zea nudivirus sequence, two additional related sequences from H. virescens and H. armigera nudiviruses for development of genetically modified nudiviruses capable of being sexually transmitted by an insect useful for controlling pest populations, methods for preparing egg populations for controlling pest populations and methods for controlling pest populations utilizing such egg populations. Such genetically modified nudiviruses are capable of causing sterility in a target population of insects. Previously disclosed were insects infected with the previously disclosed genetically modified H. zea nudivirus, methods of making genetically modified nudiviruses, and methods of using genetically modified nudiviruses to control an insect pest population.

### CROSS-REFERENCE TO RELATED APPLICATIONS

This application is a Continuation-in-part application that claims the benefit of and priority to U.S. Ser. No. 18/066,224 filed Dec. 14, 2022, entitled “Mutant Nudivirus Insect Control,” which is a Continuation-in-part application that claims the benefit of and priority to U.S. Ser. No. 16/722,071 filed Dec. 20, 2019, entitled “Compositions and Methods for Pest Control Management”; which is a Continuing application that claims the benefit and priority of U.S. Ser. No. 15/694,323 filed Sep. 1, 2017 entitled “Compositions and Methods for Pest Control Management” now Abandoned, which is a Continuing application that claims benefit and priority to U.S. Ser. No. 15/154,069 filed May 13, 2016 now U.S. Pat. No. 9,770,033 entitled “Compositions and Methods for Pest Control Management”; which claims the benefit and priority to “U.S. Provisional Application Ser. No. 62/161,674, filed on May 14, 2015 entitled “Mutant Nudivirus and Method for Using Same for Insect Control,” the contents of which all of the above are incorporated herein in their entirety for all purposes.

### REFERENCE TO AN ELECTRONIC SEQUENCE LISTING

The contents of the electronic sequence listing (Sequence Listing-Mutant Nudivirus Insect Control.xml; Size: 902,964 bytes; and Date of Creation: Dec. 14, 2022) is herein incorporated by reference in its entirety.

### BACKGROUND

Insect pests cause crop damage worldwide resulting in significant losses to food and fiber crops and increased production costs that target control of such pests. For example, the Heliothine complex of lepidopteran moths cause in excess of two billion dollars in damage and cost of control in the United States annually. While all crops are susceptible to similar pest pressure, transgenic expression of Bacillus thuringiensis (Bt) toxins was developed to control the lepidopteran pests and has become a major tool for control of these and other insect pests. Since the commercial introduction of Bt crops in 1996, they have been adopted around the world and have been grown on more than one billion acres worldwide. In the US, 81% of corn and 84% of cotton express one or more Bt toxins. (HyperTextTransferProtocol://WorldWide Web.ers.usda.gov/data-products/adoption-of-genetically-engineered--crops-in-the-us/recent-trends-in-ge-adoption.aspx, 2015 report, wherein “HyperTextTransferProtocol” is “http”, and “WorldWideWeb” is “www”.)

Unfortunately, due to the remarkable ability of insects to adapt to insecticides, resistance to Bt toxins was predicted and reports of field-evolved resistance and reduced efficacy are increasing. Such resistance is a threat to the sustainability of important Bt crops, in the U.S. and elsewhere. Thus, there is a continuing need to develop new methods to control insect pests. For example, Helicoverpa zea (H. zea, commonly known as the corn earworm), is a major polyphagous moth pest in the Heliothine complex in the United States and causes millions of dollars of damage to corn and cotton plants each year.

A number of pests in the Heliothine complex of moths, notably Helicoverpa armigera and Heliothis virescens are highly polyphagous and cause economically significant damage to many crops. Crops commonly damaged by H. armigera and H. virescens include cotton, corn, soybean, sunflowers, tomato, sorghum, strawberry, peppers, beans, aubergine, okra, peas, millet, cucumber, melon, lettuce, cauliflower, and cabbage. Because H. armigera and H. virescens attack a wide variety of plants and, in many instances, are developing resistance to Bt crops, farmers rely heavily on pesticides to control these pest insects.

The need for pest management, such as in field, fruit and vegetable crops, is a need in the art, which will only become more critical as resistance to Bt expands. Further, there is a need for pest management that does not involve the use of conventional pesticides or transgenic technologies such as in organic cropping systems. The instant invention addresses one or more aforementioned needs in the art.

Additionally, there is also a need for pest management where pest species eggs are infected with or carry pathogens to plants of which will be then protected from infestations and damage caused by feral members of the pest by spread of the pathogen. Such pest management provides where eggs distributed must contain or carry a biocontrol pathogen, such as a virus, bacteria, or fungi in which after the eggs hatch, larvae or adults that develop, carry and spread the pathogen to the feral population where the pathogen reduces feral pest numbers and vigor. This need for past management provides pathogens that ultimately reduces the wild population thereby protecting the crop and reducing the size of subsequent generations, protecting the treated crop from damage.

### BRIEF SUMMARY

Disclosed are the H. zea nudivirus sequence and two additional related sequences from H. virescens and H. armigera nudiviruses for development of genetically modified universes capable of being sexually transmitted by an insect useful for controlling pest populations. Such genetically modified nudiviruses are capable of causing sterility in a target population of insects. Previously disclosed were insects infected with the previously disclosed genetically modified H. zea nudivirus, methods of making genetically modified nudiviruses, and methods of using genetically modified nudiviruses to control an insect pest population.

Also disclosed is the use of the above sequences within applications of such sequences in the eggs of pest species where the sequences are sexually transmitted by an insect useful for controlling pest populations.

Also disclosed is applying eggs of a pest species that are infected with or carry pathogens to plants which will be protected from infestations and damage caused by feral members of the pest by spread of the pathogen. The eggs distributed must contain or carry a biocontrol pathogen, such as a virus, bacteria, or fungi. After the eggs hatch, larvae or adults that develop, carry and spread the pathogen to the feral population where the pathogen reduces feral pest numbers and vigor. This pathogen ultimately reduces the wild population thereby protecting the crop and reducing the size of subsequent generations, protecting the treated crop from damage. Embodiments rely upon the biology of the pest insect to distribute the pathogen rather than attempting to protect the crop with a broadcast spray or application of the pathogen.

An embodiment of this process is to apply the virus product with the active ingredient HzNV2 isolate 90DR71 or similar viruses with genes ORF 90, ORF 92 or PAG1 inactivated by genetic mutation, CRISPR modification or recombinant DNA methodology within infected eggs of Helicoverpa zea to corn. These eggs are applied to vegetative corn where larvae can feed and develop but do not reduce yield of the crop. The infected insects develop alongside their wild counterparts on the corn to maturity. Then as adults, the moths mate with the feral moths, sexually transmitting this sterilizing virus across the targeted pest populations. The virus infects and sterilizes offspring of these matings to reduce pest populations and protect the corn from damage.

Embodiments include the use of infected pest eggs to target and spread a biocontrol agent for control of pest populations.

Another embodiment concerns insect eggs that have been applied to plants to establish infestations of the pest and test conventional pest control methods. Pathogens have also been introduced into pest populations by release of infected arthropods to introduce and inoculate pest populations. However, the large scale release of pathogen-infected arthropods eggs for direct pest population control is novel and demonstrated within the disclosed embodiments.

A further embodiment includes applying eggs infected with or carrying a pest-pathogen that can spread across targeted pest populations. The eggs must lead to the spread of a biocontrol pathogen in the wild population. The eggs may be applied directly in a spray or by release of females that will lay pathogen-infected eggs that will spread the pathogen to targeted pests.

An embodiment entails release of the biocontrol pathogen within or on pest eggs. Eggs are hardier than moths, easily produced in laboratory production facilities and can be synchronized with targeted pest populations. Transporting and shipping eggs is easier due to their small size, and they can be stored prior to use. The eggs can be easily spread across the field with conventional spraying equipment and can be formulated to reduce predation. Releasing eggs also has the potential benefit of synchronizing emergence of the adult infected moths with adult wild moths, as they will be developing in the same conditions. This maximizes chances for the infected moths to mate with the wild moths. Producing eggs is faster and easier than producing adult moths, reducing the cost, labor and space requirements involved in preparing to spread the pathogen, as each adult female can produce many eggs.

The infected pest insect spreads the pathogen within the targeted pest population. They will find and infest the same parts of the plant. These biological pathogens have evolved systems for spreading within populations and this invention takes advantage of those evolutionary systems for distribution while manipulating the basal rate of pathogen infection. Natural pathogens are known to efficiently control pest populations under some conditions. This invention provides for control of the infestation level of a pathogen which is the key factor for suppressing pest populations.

In another embodiment the timing of application of the infected insect egg can be selected to reduce crop damage of the application while maximizing its potential to reduce populations of feral pests. The basis of applying pathogen infected eggs cannot be changed. However, the means of how the eggs are applied, what solution they are applied in, or what the target pest or biocontrol agent is, has flexibility.

In an embodiment the insect being targeted is limited only by the ability to infect that insect with a biocontrol pathogen, and to produce eggs at a scale large enough to support the applications. The pathogen utilized could reduce population sizes in multiple ways, such as causing immediate sterilization, sterilizing the next generation, or causing insect mortality.

The way the eggs are applied to the plants can be performed in several ways without changing the method—this could include manual, mechanical, or aerial deployment of eggs onto the plants. The eggs could be applied by hand in very small areas, though this may be used less in practice due to the need for wider scale applications. For larger areas, a spray or aerial application could be used to cover a wide area at one time.

In another embodiment, the eggs could be applied in formulations that enhance the application. It is contemplated that the solution with the eggs could contain a sticking agent, such as sucrose-flour or sucrose-starch. The solution could contain a surfactant such as dishwashing detergents to encourage eggs to stay in suspension, rather than floating and clumping. The specific gravity of the solution may be controlled so that the eggs are isotonic and remain in solution rather than settle. The solution may contain antifeedants that repel arthropods that normally feed on pest eggs. The pathogen-infested eggs could also be delivered by adult insects that transmit the pathogen to progeny.

The basis of applying pathogen infected eggs cannot be changed. However, the means of them spreading the pathogen could be altered. The best method described indicates the developing larvae within the eggs are already infected with the pathogen, but another way of achieving the same effect could be coating the eggs so that the larvae pick up the pathogen immediately upon emergence.

A further embodiment is the formulation of a solution which the eggs are suspended in for application could be produced in a way that protects the eggs from mechanical damage during spraying, encourage them sticking on the target plants, prevent egg clumping, to serve as additional nutrients for hatching larvae, to discourage egg predation, or other purposes that would enhance the efficacy of the technique. If needed for mechanical applications, a specialized nozzle or reservoir could be developed to further prevent egg clumping or damage upon spraying.

Ideal concentration of eggs to be applied to the plants can be optimized to ensure sufficient survival of the insects to spread the pathogen. The formulation may potentially be left out in some situations. What ingredients it would include may differ by crop treated, location, weather, etc.

The disclosed embodiments can be used for research purposes, potentially using pathogens that are under investigation but may not be currently demonstrated to be useful for biocontrol.

The disclosed embodiments must take into consideration environmental and pathogen-specific factors to work to their full capacity. It is contemplated that if the eggs are being sprayed in conditions that are not suitable for survival of emerging larvae (ex. chemical pesticide exposure the emerging larvae are not resistant to) or pathogen, the invention may not have its intended effect. The eggs must produce insects capable of spreading a pathogen.

Conventional methods to achieve control include releasing adult sterile moths into crops when wild adult moths are present in the field. There has yet to be a means of releasing immature insects or eggs for the sterile insect technique. Additionally, the application of insect eggs to crops for biocontrol has only been performed using the eggs of natural enemies to the pest species, not eggs of the pest species itself. Pathogens targeting larvae, (e.g., entomopathogenic fungi) have also been sprayed on plants to infect the wild populations.

While these methods may involve use of a pathogen, or application of insect eggs, none of these methods use the insect eggs of the pest species itself to lead to the targeted spread of a pathogen in wild populations.

### BRIEF DESCRIPTION OF THE DRAWINGS AND FIGURES

FIG. 1. Schematic of the model Helicoverpa zea nudivirus 2 (HzNV-2) genome showing genes known to be involved or implicated in sterilizing mutations.

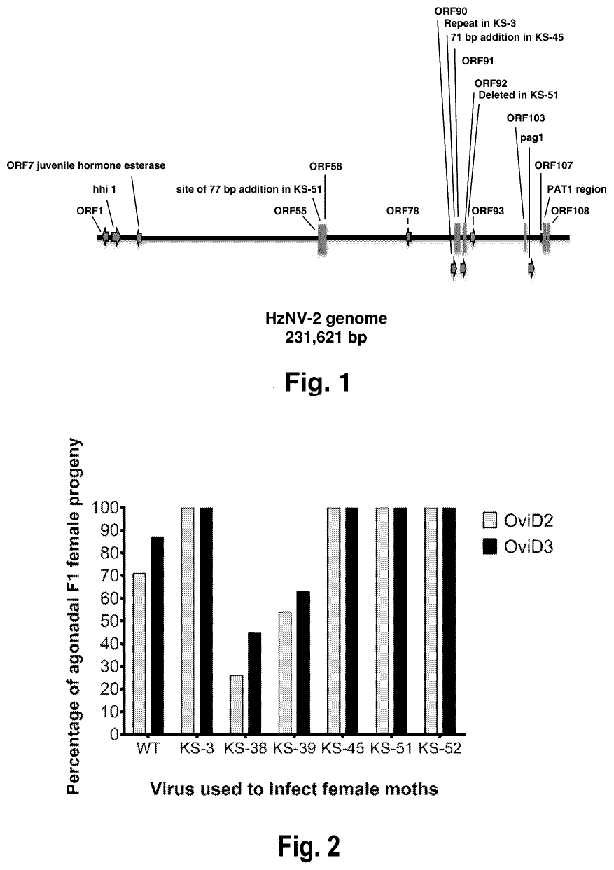

FIG. 2. Percentage of agonadal female F1 progeny. Wild-type (WT) and mutant HzNV-2 generated by chemical mutagenesis (KS-3, KS-38, KS-39, KS-45, KS-51, and KS-52) were injected into adult female moths on the day of emergence, and eggs were collected on oviposition days 2 (OviD2) and 3 (OviD3). Female F1 progeny were reared to adult moths and evaluated for the presence of a viral plug indicating an agonadal pathology (OviD2-WT n=17, KS-3 n=24, KS-38 n=23, KS-39 n=26, KS-45 n=19, KS-51 n=14, KS-52 n=18; OviD3-WT n=15, KS-3 n=16, KS-38 n=22, KS-39 n=16, KS-45 n=19, KS-51 n=18, KS-52 n=23).

FIG. 3. Occurrence of complete sterility in agonadal female F1 progeny. Wild-type (WT) and mutant HzNV-2 (KS-3, KS-38, KS-39, KS-45, KS-51, and KS-52) were injected into adult female moths on the day of emergence, and offspring eggs were collected on oviposition days 2 (OviD2) and 3 (OviD3). Female F1 progeny were reared to adult moths and evaluated for ability to lay viable eggs. Failure to lay eggs or lay viable eggs indicates complete sterility (OviD2-WT n=17, KS-3 n=24, KS-38 n=23, KS-39 n=26, KS-45 n=19, KS-51 n=14, KS-52 n=18; OviD3-WT n=15, KS-3 n=16, KS-38 n=22, KS-39 n=16, KS-45 n=19, KS-51 n=18, KS-52 n=23). This illustrates that these mutant HzNV-2 causes decreased egg production and sterility.

FIG. 4. Effect of viral titer on H. zea egg production. Female adult moths (7) were injected with 100 μl wt HzNV-2 (high dose: 4×104 pfu/ml; low dose: 40 pfu/ml) or yfp recombinant HzNV-2 (high dose: 1×107 pfu/ml; low dose: 1×104 pfu/ml) isolated from cell culture and mated with uninfected male moths (9). Eggs were collected on oviposition days 2-5 and counted. Uninfected females were mated with uninfected males as a control.

FIG. 5. Direct inoculation of insect larvae causes high numbers of agonadal moths. Wild-type (WT) HzNV-2 and mutant KS3 were amplified in Sf9 insect cell culture and injected into third instar larvae via an insulin syringe. WT HzNV-2 and mutant yfp HzNV-2 virus isolated from viral plugs of agonadal female moths were used to infect third instar larvae via a pre-sterilized pin. After both types of infections, larvae were reared to adult moths, and females were examined for the presence of a viral plug, an indicator of virus infection and agonadal pathology (Syringe, WT n=20, KS3 n=25; Pin, WT n=20, yfp HzNV-2 n=21).

FIG. 6. 1.5% agarose gel showing PCR results that yfp HzNV-2 is a pag1 mutant. Wild-type (WT) HzNV-2 (control) and yfp HzNV-2 viral DNA and yfp-pUC57 (control), the plasmid used for homologous recombination to make the yfp HzNV-2 virus, were used as DNA templates in the PCR reactions. If present, yfp primers were used to amplify the yellow fluorescent protein gene (547 bp); pag1 primers were used to amplify pag1 DNA; ORF78 primers were used to amplify hypothetical gene ORF78 (403 bp) that the DNA is from HzNV-2.

FIG. 7 Schematic alignment of HvNV genome with HzNV-2 genome.

FIG. 8. Schematic alignment of HvNV ORF 90 with HzNV-2 ORF 90.

FIG. 9. Schematic alignment of HvNV ORF 92 with HzNV-2 ORF 92.

FIG. 10 Schematic alignment of HaNV with HzNV-2 genome.

FIG. 11. Schematic alignment of HaNV ORF 90 with HzNV-2 ORF 90.

FIG. 12. Schematic alignment of HaNV ORF 92 with HzNV-2 ORF 92.

FIG. 13A-13D. Together show the whole genome alignment of HvNV main contig and HzNV-2 genome.

FIG. 14 Schematic alignment of HvNV pag1 genome with HzNV-2 pag1 genome.

FIG. 15 Schematic alignment of HaNV pag1 genome with HzNV-2 pag1 genome.

Brief description of Sequence ID's of nudivirus genomic and amino acid sequences

Sequences ID1-19. Sequences of Heliothis zea nudivirus (HzNV) genome. These sequences were originally filed in U.S. patent application Ser. No. 15/154,069 filed May 13, 2016, now U.S. Pat. No. 9,770,033 issued Sep. 26, 2017, from which the current application depends. Sequences of U.S. Pat. No. 9,770,033 are inserted as required by WIPO ST.26 protocol whereby the sequences of the earliest priority application are disclosed as required.

Sequence ID20. Sequence of Heliothis virescens nudivirus (HvNV) genome.

Sequence ID21. Predicted peptide sequence of HvNV ORF 90

Sequence ID22. Predicted peptide sequence of HvNV ORF 92

Sequence ID23. Partial sequence of Heliothis armigera (HaNV) genome.

Sequence ID24. Predicted peptide sequence of HaNV ORF 90

Sequence ID25. Predicted peptide sequence of HaNV ORF 92.

Sequence ID26. Sequence of HvNV pag1 nucleotide sequence.

Sequence ID27. Sequence of HaNV pag1 nucleotide sequence.

### DETAILED DESCRIPTION

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “closely related” as used herein, with respect to the term insect and/or moth, means a species so closely related so as to support replication of the HzNV-2, HvNV or HaNV viruses.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed”. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “gene”, also called a “structural gene” means a DNA sequence that codes for or corresponds to a particular sequence of amino acids which comprise all or part of one or more proteins or enzymes and may or may not include introns and regulatory DNA sequences, such as promoter sequences, 5′-untranslated region, or 3′-untranslated region which affect for example the conditions under which the gene is expressed. Some genes, which are not structural genes, may be transcribed from DNA to RNA, but are not translated into an amino acid sequence. Other genes may function as regulators of structural genes or as regulators of DNA transcription.

By “genetically modified” is meant a gene that is altered from its native state. The term “genetically modified,” as used herein, includes a sequence (a virus, for example) that contains genetic material from more than one organism. The term further includes a sequence that is modified from its native state, for example, via a deletion or insertion, and which does not include genetic material from more than one organism. The latter may be referred to as a “mutant” as used herein.

The instant disclosure addresses one or more needs in the art as described above. In one aspect, the present disclosure addresses the globally important need for new methods to control insect pests in crops threatened by such pests. In a further aspect, the disclosure addresses an increasingly important issue, Bt resistance, that threatens the sustainability of insect-resistant transgenic crops.

A sexually transmitted insect virus, Helicoverpa zea nudivirus 2 (HzNV-2, accession number NC_004156.), is known to cause approximately 33% of infected H. zea to be sterile. (Raina 1995). Wildtype (WT) HzNV-2, however, is not a potential biological control agent due to the high proportion of asymptomatic carrier moths. Applicant has found that HzNV-2 can be modified so that extremely high percentages, for example, up to 100%, or greater than about 90%, of the infected H. zea become sterile (U.S. Pat. No. 9,770,033). This invention describes two novel nudiviruses, the Heliothis virescens nudivirus (HvNV) and the Helicoverpa armigera nudivirus (HaNV) that enable genetic modification as described in the patent applications U.S. Ser. Nos. 16/722,071 and 15/154,069 directed to HzNV-2 to which this invention claims priority. In these priority applications, HzNV-2 is shown to be an important tool in controlling various insect pests by causing collapse in the target insect population.

As such, these two-novel mutant nudiviruses, HvNV and HaNV, may be an important tool in controlling various insect pests by causing collapse in the target insect population. In turn, this modified virus can be used to infect a target insect and control an insect population without the use of traditional pesticides, or, alternatively, can be used in combination with traditional pesticides such that the amount of the pesticide used is minimized. Such a technology may have utility in control of populations of Bt resistant insects and invasive insect populations for which traditional pesticides are ineffective. Applicant's approach allows for pest control via release of insects infected with a sexually transmitted virus that can be transmitted by mating in the targeted farming area. The approach developed by Applicant is effective for both transgenic and/or non-transgenic crops and can target pest species in which the virus replicates and is sexually transmitted.

Attempts to control insect populations via genetic manipulation of crops is currently limited due to the ability of insects to rapidly develop resistance to the genetically added toxins and is further limited by the costs to producers to use such modified crops. For example, crops expressing the Bacillus thuringiensis (Bt) toxins were introduced twenty years ago to control caterpillar pests. Since then, they have been adopted worldwide, planted on more than one billion acres, and have become one of the most successful and rapidly adopted agricultural technologies since the ‘green revolution’ of the mid-20th century (James, 2012). As of 2015 in the US, 81% of corn and 84% of cotton express one or more Bt toxins. However, the widespread adoption of Bt technology carries the significant risk that overuse will inevitably lead to development of insect resistance to Bt toxins and crop failures, which threatens the technology's continued viability (Carriere et al., 2010, Tabashnik et al., 2013; Tabashnik et al., 2009). This risk, which has always been recognized by regulators, industry, and researchers, has been managed by resistance monitoring and the use of refuge strategies to delay resistance. These refuge strategies, which have been mandated by the EPA in the USA with similar mandates in other countries, entail the planting of nearby non-transgenic plants to maintain susceptible insect populations (EPA, 1998; Huang et al., 2011). Unfortunately, this practice is not always followed due to cost to producers and is not always effective because of the remarkable ability of insects to evolve resistance to insecticides. Increasing insect resistance to Bt plants is reported, and some insects exhibit resistance traits that are genetically dominant (Campagne et al., 2013). To summarize, Bt-resistant insects represent an ongoing and increasingly important threat to the continued efficacy of Bt crops, and to food and fiber production in the US and worldwide.

One insect pest threatening Bt crops is the corn earworm, Helicoverpa zea, a lepidopteran moth. H. zea is found throughout North America, for example, where it is the second most costly crop pest (Fitt, 1989), and is also found in Central America, the Caribbean, and South America. H. zea, which feeds on many different plants and has several common names (e.g., corn earworm, cotton budworm, tomato fruitworm) has some strains that are 1000-times more resistant to Bt toxin than susceptible insects (Ali and Luttrell, 2007; Ali et al., 2006).

Applicant has developed a new approach to suppress insect pest populations. In a further aspect, Applicant has developed a new approach to managing Bt resistance, which relics upon engineering or mutating a sexually transmitted insect virus that sterilizes infected insects (including complete or partial sterility). Insects containing the mutant virus may be released in areas where Bt resistance is present in H. zea populations, thereby suppressing these targeted populations and preserving the utility of the Bt transgenic plants and/or non-transgenic plants. Similarly, the susceptible pest insects are commonly invasive across the world and the disclosed methods may be used to reduce and eliminate the invasive insect pest populations. The viruses developed by Applicant are mutant and recombinant forms of a naturally occurring (i.e., wild-type) virus, Helicoverpa zea nudivirus 2 (HzNV-2), which infects H. zea. HzNV-2 was previously the only lepidopteran insect virus which has been shown to be sexually transmitted and causes sterility in both male and female moths. In one aspect, the infected insect may have partial sterility, defined as when a female H. zea moth lays less than 30 viable eggs each day due to damage to her reproductive organs. In one aspect, the infected insect may have complete sterility, defined as the inability of a female moth to lay viable eggs due to damage to her reproductive organs.

In one aspect, disclosed is a genetically modified nudivirus of a wild type nudivirus for HzNV-2, HvNV and HaNV. The genetically modified nudivirus contemplated herein is generally capable of being sexually transmitted by an insect and capable of causing sterility in an insect at a rate of greater than about 50%, or from about 50% to about 100%, or from about 80% to about 95% or from about 90% to about 100% following infection of said insect with said nudivirus comprising a genetic mutation.

In one aspect, the wild type nudivirus has at least about 80% sequence identity to Helicoverpa zea nudivirus 2 (HzNV-2) virus. The wild type nudivirus may be characterized in that it has a latent phase, has about 80% or greater sequence identity to Helicoverpa zea nudivirus 2 (HzNV-2, also known as Heliothis zea nudivirus or gonad specific virus) virus, and is capable of replicating in one or more moths.

In one aspect, the genetically modified nudivirus HzNV-2 and related nudivirus for HzNV-2, HvNV and HaNV may contain a disruption in a latent phase of said wild type nudivirus.

In one aspect, the insect may be a lepidopteran moth in the family noctuidae which supports replication of the HzNV-2 virus in reproductive tissues sufficient to cause sterility at a rate of 50% or greater. The insect may be selected from Helicoverpa zea (H. zea) H. armigera, Heliothis virescens, H. assulta, Agrotis ipsilon, Spodoptera frugiperda, Spodoptera exiguae, closely related moths, noctuid moths, or combinations thereof.

In one aspect, genetic modification may be a mutation in one or more genes selected from the HzNV-2 persistence-associated gene (pag1) (which encodes PAT1, or the persistence associated transcript (SEQ ID NO: 7)), ORF 90 (SEQ ID NO: 4), ORF92 (SEQ ID NO: 5), ORF 2 (SEQ ID NO: 3), or combinations thereof, such that the modification is sufficient to disrupt expression of one or more of such genes, for example, wherein said disruption reduces expression or is a functional knockout. In one aspect, the genetic modification may be a mutation in the HzNV-2 persistence associated gene (pag1) sufficient to disrupt expression of the pag1 gene. In one aspect, the genetic modification may be a mutation in the HzNV-2 pag1 gene (SEQ ID NO: 7), which is the persistently associated transcript and has been shown to be involved in the establishment of latent infections of HzNV-1. In one aspect, the genetic modification may be a mutation in one or more sequences selected from HzNV-2 dr1 (atgaagctgaggatgaatctgaac, SEQ ID NO: 14), dr2 (gaaactcctaaatcaaaggatgaacctaaagcaaag, SEQ ID NO: 15), dr3 (atgaaaaagcaaaggctgaggcgaaggctaaagccgatgctgctgcaaaagccaaagctg, SEQ ID NO: 16), dr4 (ttataccagagagcaagccagaaa, SEQ ID NO: 17), dr5 (acctaaagttgaatctaaagtagt ggaaccacctaaagcggaatctaa aacagtggaagctcctactaaaacagttgaagt, SEQ ID NO: 18), dr6 (agctgccgctaaacgcaaagccgaggctga, SEQ ID NO: 19), or a combination thereof. In one aspect, the genetic modification may be a mutation in HzVN-2 dr3 (SEQ ID NO: 16), for example, KS-3 in which there is a bp insertion at 175,550 and KS-45, 80 bp insertion at 175,650. In one aspect, the genetic modification may be a mutation in HzNV-2 dr6 (SEQ ID NO: 19), for example, KS-51, having a 29 bp delction at 180,270-180,299.

While the direct repeat sequences disclosed here in the HvNV or HaNV nudivirus genomes are not identical to HzNV-2, similar structural repeats in baculovirus genomes may vary in nucleotide sequence or number but retain functionality for regulating viral gene expression or viral DNA replication. An example of such repeats are the homologous repeats of Autograph californica nucleopolyhedrosis virus and insertion of transposable elements that vary in sequence but alter baculovirus gene expression and replication. Similar functional roles for nudivirus repetitive sequences would be expected by one of ordinary skill in art in view of references such as, for example: Carstens EB, Wu Y. No single homologous repeat region is essential for DNA replication of the baculovirus Autographa californica multiple nucleopolyhedrovirus. J Gen Virol. 2007 January; 88(Pt 1):114-122. doi: 10.1099/vir.0.82384-0. PMID: 17170443; and Blissard GW, Rohrmann GF. Baculovirus diversity and molecular biology. Annu Rev Entomol. 1990; 35:127-55. doi: 10.1146/annurev.en.35.010190.001015. PMID: 2154158.

In a further aspect, the genetic modification is one in which an increase in activity of a viral regulatory gene results from the modification, wherein said viral regulatory gene is HzNV-2 hhi-1 (SEQ ID NO: 6). In certain aspects, the identification of a genetic modification of interest can be determined via detection of increased hhi-1 activity.

In one aspect, the genetically modified nudivirus, may be obtained via chemical mutagenesis. In another aspect, the genetically modified nudivirus may be obtained via recombinant DNA technology. Exemplary, non-limiting methods are disclosed herein. In a further aspect, the genetically modified nudivirus may be obtained using gene editing technology as is known in the art.

In one aspect, a method of reducing a population of lepidopteran moths is disclosed. The method may comprise the step of introducing an insect infected with a genetically modified nudivirus as disclosed herein into the population of interest. In one aspect, infected insects of a single sex may be introduced into a target population, for example an all-male or all-female population of insects. In another aspect, a mixed sex population of infected insects may be introduced.

In one aspect, an insect infected with a virus as described above is disclosed. The insect may be a lepidopteran moth. In further aspects, the insect may be Helicoverpa zea (H. zea) H. armigera, H. assulta, Heliothis virescens, Agrotis ipsilon, Spodoptera frugiperda, Spodoptera exiguae or a closely-related moth, or noctuid moths. The insect may be a female or a male.

In one aspect, a method of making an insect capable of transmitting a genetically modified nudivirus as disclosed herein to a population of insects is disclosed. The method may comprise the step of infecting an insect with a genetically modified nudivirus as described herein. In one aspect, the insect is a lepidopteran moth. The insect may be Helicoverpa zea (H. zea) H. armigera, H. assulta, Heliothis virescens, Agrotis ipsilon, Spodoptera frugiperda, Spodoptera exiguae or a closely related moth or noctuid moth. The method may utilize male insects, female insects, or both. In one aspect, the genetically modified nudivirus may be derived from a viral plug. The genetically modified nudivirus may be administered orally to the insect. In other aspects, the genetically modified nudivirus may be administered to an insect via direct inoculation of insect larvae or adult moths by puncturing the cuticle of the insect with a pin containing viral inoculum derived from a viral plug. In a further aspect, the genetically modified nudivirus may be administered to the insect via direct hypodermic injection into third instar larvae or moths.

In one aspect a closely related moth is functionally defined as those which support virus replication and exhibit reproductive tract pathology when infected by HzNV-2, HvNV or HaNV.

In one aspect, a method of protecting a crop susceptible to a moth pest from moth pest damage is disclosed. In this aspect, the method may comprise the step of introducing insects infected with a genetically modified nudivirus as described herein, into a crop of interest. The crop may be any crop threatened by the pest, and may include, for example, the following non-limiting list of crops: corn, cotton, soybeans, tomatoes, sorghum, artichoke, asparagus, cabbage, cantaloupe, collard, cowpea, cucumber, eggplant, lettuce, lima bean, melon, okra, pea, pepper, potato, pumpkin, snap bean, spinach, squash, sweet potato, and watermelon, alfalfa, clover, cotton, flax, oat, millet, rice, sorghum, soybean, sugarcane, sunflower, tobacco, vetch, and wheat, avocado, grape, peaches, pear, plum, raspberry, strawberry, carnation, geranium, gladiolus, nasturtium, rose, snapdragon, zinnia, and combinations thereof. (sec//edis.ifas.ufl.edu/in302) In certain aspects, the crop may be a Bacillus thuringiensis (Bt) toxin producing crop. The insect used may be any insect as described above.

In a further aspect, a method of sterilizing an insect population is disclosed. The method may include the step of introducing a genetically modified nudivirus as described herein into a target insect population. This may include an invasive insect population, and may further include an insect population that is Bt resistant. One such example insect is the lepidopteran moth, which may further include Helicoverpa zea (H. zea), H. armigera, H. assulta, Heliothis virescens, Agrotis ipsilon, Spodoptera frugiperda, Spodoptera exiguae and closely related moths or noctuid moths.

In one aspect, a method of making a genetically modified nudivirus via chemical modification is disclosed. The method may comprise the steps of
a) incubating a population of insect cells infected with a virus with about 0.05 mM to about 0.1 mM 1,3-butadiene diepoxide (or 1,2,3,4-Diepoxybutane or “DEB”) for at least 1 hour and up to five hours at a temperature range of about 26 to about 28° C., wherein the population of infected insect cells may comprise an Sf9 insect cell, for example, further wherein the insect cell may be infected with a virus having at least 80% identity, or at least 85% identity, or at least 90% identity, or at least 95% identity, or at least 99% identity to a wild-type HzNV-2 virus (SEQ ID NO: 1), and wherein the incubation is sufficient to induce one or more mutations in the HzNV-2 virus;
b) purifying the virus, wherein the purifying step includes the steps of
i. culturing the population of insect cells infected with a virus in a DEB-free media, wherein the population of infected insect cells are isolated and washed prior to the culturing step;
ii. collecting a supernatant from DEB-free media to obtain a DEB-exposed virus population;
c) amplifying and collecting the mutated virus from the DEB-exposed virus population, wherein the collection step may comprise selecting virus from a plaque having a large plaque phenotype.

In another aspect, a new approach has been developed to introduce pathogens to target arthropod pest populations by delivering the pathogen within or on pest eggs. This new development allows for manual or mechanical distribution of eggs that carry the pathogen, or the pest itself, to disseminate the pathogen throughout targeted pest populations.

It is known that biopesticides such as viral, fungal or bacterial pathogens of arthropods are conventionally produced in manufacturing centers then delivered to pest populations by spraying the pathogen on to plants. Conventional distribution methods spread the pathogen across the environment where most of the product never encounters the targeted pest and is subject to inactivation by environmental factors such as desiccation and ultraviolet light. Such conventional broadcast distribution method contributes to reduced efficacy of many biopesticides.

In view of the above, the new approach applies the pathogen within or on eggs of the arthropod pests for the purpose of spreading the pathogen from infected to uninfected pests, thereby reducing targeted pest populations and protecting crops. This new approach involves producing eggs infected with or carrying a biocontrol pathogen such that insects hatching from these eggs become infected with the pathogen and distribute the pathogen across targeted pest populations.

In one aspect of the new approach, viral pathogens target the larval stage.

Baculoviruses are widely used for pest control and are sprayed onto crops. The baculovirus pesticide can be formulated and used to coat the surface of insect eggs so that larvae become infected as they emerge from the egg. These infected larvae distribute the pest within targeted populations before they die.

In another aspect of the new approach is where entomopathogenic fungi are also sprayed on to crops where most do not come into contact with target pests. In a similar approach, fungi can be formulated to coat the surface of insect eggs where it is spread by the emerging insect to targeted pest populations.

In yet another aspect of the new approach is viral pathogens targeting the adult insect. In this aspect, the pathogen is carried by the larvae until the adult stage where it is transmitted between adult moths. Adult moths that emerge from the applied eggs will then spread the infection to feral moths, through mating or other means. This biocontrol pathogen will then reduce wild moth populations, such as by producing sterile adults. Sterile adults lead to the reduction of the subsequent generation, reducing crop damage.

In the above aspects of the new approach of introducing pathogens to target arthropod pest populations by delivering the pathogen within or on pest eggs, the application of these eggs could be manual, mechanical, or aerial. The eggs in such aspects may or may not be combined with a formulation to aid the process (e.g., to protect the eggs from physical damage or predation, to encourage sticking on the plants, prevent egg clumping, to serve as additional nutrients for hatching larvae, or other purposes).

In an alternative approach to these aspects, where pathogens that are transmitted vertically from parents to progeny, infected adult arthropods are released into fields where female oviposition will distribute the pathogen-carrying eggs. The pathogen then infects feral pest arthropods and thereby protect the crop from damage.

Exemplary methods making a genetically modified nudivirus via chemical modification are provided below. Further, exemplary methods for introducing pathogens to target arthropod pest populations by delivering the pathogen within or on pest eggs are provided further.

### EXAMPLES

The present invention may be understood more readily by reference to the following detailed description of preferred embodiments of the invention and the Examples included therein and to the Figures and their previous and following description. Although any methods and materials similar or equivalent to those described herein can be used in the practice or testing of the present invention, the preferred methods, devices, and materials are now described. All references, publications, patents, patent applications, and commercial materials mentioned herein are incorporated herein by reference for the purpose of describing and disclosing the materials and/or methodologies which are reported in the publications which might be used in connection with the invention. Nothing herein is to be construed as an admission that the invention is not entitled to antedate such disclosure by virtue of prior invention.

### Method—Generating a Recombinant HzNV-2 Virus

To generate a yfp insert mutant virus using recombinant DNA technology, Applicant replaced the pag1 gene with a gene encoding yellow fluorescent protein (yfp) by homologous recombination. pag1 expresses a microRNA that suppresses the expression of the viral transcription factor, hhi-1, an RNA intermediate necessary to maintain latency in HzNV-1 (Chao, 1998; Wu and Wu, 2011). To inactivate the pag1 gene, a pUC57-based transfer vector, yfp-pUC57, was designed and synthesized with the yfp gene controlled by the Orgyia pseudotsugata multicapsid nuclear polyhedrosis virus immediate early 2 (OplE2) promoter, and flanked by 1.2 kb of viral HzNV-2 sequences upstream and downstream of pag1.

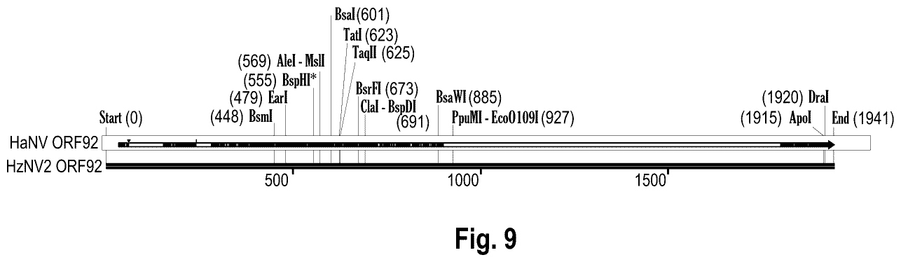

The yfp HzNV-2 recombinant virus was generated by homologous recombination of yfp/pag1-pUC57 plasmid with WT HzNV-2 genomic DNA after transfection into Sf9 insect cells. The mutant virus was plaque purified, screened for YFP fluorescence using a Zeiss observer A1 fluorescent microscope and the AxioVision Rel. 4.6 program, and amplified in Sf9 cells in nudivirus media (1× Supplemented Grace's Media, 7% FBS, 1% Penicillin/streptomycin) in 25 cm² tissue culture flasks. Viral DNA was isolated from the cell culture supernatant using DNAzol (ThermoFisher Scientific #10503027) and PCR was performed using the AmpliTaq Gold master mix (ThermoFisher Scientific #4398881). PCR results confirmed deletion of pag1 and presence of the yfp gene using internal pag1 (F 5′-GTGGTGCCAGACTTTCAGACATCAT-3′ (SEQ ID NO: 10), R 5′-GGGTCTGTTGCGACCTAAAGGTCTA (SEQ ID NO: 11)) and yfp (F 5′-CGAAGAGCTCTTCACTGGCGTGGT-3′ (SEQ ID NO: 12), R 5′-GGTGTTTTGCTGGTAATGATCCGC-3′ (SEQ ID NO: 13)) primers, respectively (FIG. 6). Subsequent PCR reactions detected pag1 DNA indicating that the yfp HzNV-2 virus is a yfp insertional mutant rather than a complete replacement of this gene. Nevertheless, inactivation of pag1 was sufficient to produce a virus that caused sterility in up to 100% of infected insects. Primers to the HzNV-2 open-reading frame ORF78 (F 5′-GCACACCTATCGATCACCAT-3′ (SEQ ID NO: 1 positions 152572 to 152591), R 5′-GCACGATTCGTAATGTTCAAGG-3′ (SEQ ID NO: 2 positions 81062 to 81083)) were used as a control for detecting the HzNv-2 genome.

### Method of Targeting Mutations in a Nudivirus Genome

Applicant has developed a novel method using diepoxybutane (DEB) to produce deletions in nudivirus genomes by chemical mutagenesis. DEB is known to crosslink DNA and lead to deletions of multiple bases (from ˜50 bases to several kilobases), often within a single gene (Reardon et al., 1987; Wijen et al., 2001). DEB tends to cause mutations within regions of DNA that are actively transcribed. However, published protocols for DEB mutagenesis do not teach mutating a nudivirus as it infects an insect cell so that genes involved in establishing viral lysogeny are preferentially mutated. In the literature, DEB mutagenesis usually involves feeding DEB to insects (Reardon et al., 1987, Genetics 115:323-331; Kimble et al., 1990 Genetics. 1990 December; 126(4):991-1005; Olsen and Green 1982 Mutat Res. 1982 Feb. 22; 92(1-2):107-15), or exposing DNA directly to the mutagen (Yazaki et al., J Virol Methods. 1986 November; 14(3-4):275-83). See also Gherezghiher et al. (J Proteome Res 2013, 12(5):2151-2164), Kempf et al. (Biosci Rep 1990 10(4):363-374), “Methods of identifying anti-viral agents” U.S. Pat. No. 7,476,499), which relate to chemical mutagenesis, but which do not describe methods for targeting viral mutations to selective classes of viral genes as disclosed herein. The art does not teach the use of DEB to mutate nudivirus genomes during an infection or methods to target viral genes by synchronizing chemical exposure with stages of the virus infection process in cells. Disclosed herein is a novel method that addresses one or more of the following objectives: 1) efficient mutation in early expressed genes in the HzNV-2 genome, 2) introducing a mutation while avoiding viral DNA damage to a level that compromises virus replication, 3) introducing a mutation while avoiding killing the virus-infected host Sf9 insect cell, and 4) allowing recovery of virus mutants before host cells lyse and the virus becomes unstable (typically in less than 48 h).

Applicant established an efficient protocol, wherein Sf9 cells were infected with WT HzNV-2 at a multiplicity of infection (MOI) of 1 for 1.5 hours. The 1.5 hour incubation time was chosen because previous literature illustrated that 2 hours was enough time for the related HzNV-1 virus (SEQ ID NO: 2) to enter Sf21 insect cells and to transcribe the pag1 gene (Chao et al., 1992. J Virol 66(3):1442-1448). Applicant found that one barrier to an effective method was allowing sufficient time for the virus to enter into the target cell (the insect cell) and start viral transcription. Without intending to be limited by theory, Applicant found that about 1.5 hours was sufficient to achieve this objective. In other aspects, the infection time may be about 45 minutes or more, or about one hour to about two hours. Following this step, 0.1 mM DEB is added to the culture. Applicant found that, at higher concentrations of DEB (equal to and greater than about 0.5 mM), the host cell (for example, Sf9 cells) could not survive. A range of from about 0.05 mM to about 0.1 mM DEB is considered sufficient to carry out the protocol. The cells were harvested by centrifugation after a three hours incubation and re-suspended in fresh medium. Applicant found that three hours was sufficient to cause mutagenesis but not kill host insect cells. In other aspects, the infection time may be about four to five hours.

Plaque assays are performed to isolate mutated viruses, which are then amplified in Sf9 cells and evaluated for cell lysis. A mutation in a locus required for the virus latent phase, such as the pag1 region, should result in increased cell lysis because infected cells do not enter a latent phase. Increased lysis of virus-infected cells could be evident from observations of viral plaque morphology in which lytic virus mutants were larger than wild type virus plaques. Following the protocol, Applicant found that almost half of DEB-treated viral plaques (26 of 66) had a large plaque phenotype. The plaques may be preserved as DEB-mutant HzNV-2 viral stocks. 31 DEB-treated viruses were further screened to determine if they caused agonadal female moths. Briefly, third instar larvae were inoculated with a pre-sterilized pin dipped in mutant viral supernatants collected from cell culture. Larvae were reared to adults, and female moths were evaluated for the ability to lay eggs and for the presence of a viral plug. Applicant found that 11 of the 31 mutants (KS-3, KS-38, KS-39, KS-40, KS-45, KS-49, KS-50, KS-51, KS-54, KS-57, KS-65) caused a higher percentage of agonadal females than WT virus and one virus (KS-52) led to lower egg production. The outcome of the DEB mutagenesis was surprisingly efficient; 36% of the 30 viruses caused more agonadal females than the WT virus in the in vivo screen. Random mutation of the HzNV-2 genome would be expected to rarely affect latency because only two of more than 113 viral genes are known to have a role in establishing the latency. In the described method, however, approximately ⅓ of mutants appeared to alter or eliminate the latent phase.

A detailed exemplary method is as follows:

Culture conditions. Seed Sf9 insect cells at 2.5×10⁶ cells/ml in 2 mL with nudivirus media (1× Supplemented Grace's Media, 7% FBS, 1% Penicillin/streptomycin) in a 25 cm² tissue culture flask. Then 3 mL wild-type HzNV-2 virus previously amplified in tissue culture at an estimated MOI of 1 is added.

WT HzNV-2 virus was amplified by first seeding Sf9 insect cells in all wells of a 6-well culture dish at 8×10⁵ cells/ml in 2 ml nudivirus media. After a 1 hour incubation at 27° C., 50 μl of filtered WT HzNV-2 obtained from a viral plug of agonadal female moth (acquired the same day) was added to each well using a large bore tip. Plates were incubated for 2 days at 27° C. Viral supernatants were then collected, cells and debris were removed by centrifugation (900×g, 10 minutes, 4° C.), and supernatants from all wells were filter sterilized using a 0.22 μm filter and combined. The approximate viral titer from the procedure is 1.5×10⁶ pfu/ml.

To isolate the WT HzNV-2 from infected female moths, the viral plug from an infected female moth is first extracted from the body and moved to a 1.5 ml-microcentrifuge tube. One hundred (100) μl 1× PBS is added and the plug is homogenized manually with a pipette tip to release the virus. The large fragments of insect cuticle and tissue are then removed. This viral solution is termed unfiltered viral plug extract (UVPE). The filtered viral plug extract (FVPE) is a filtered solution (with a 0.22 μm filter) of 1× supplemented Grace's media, 2% unfiltered viral plug extract, and 5% penicillin/streptomycin antibiotics.

Controls for this mutagenesis are performed in parallel. Controls included uninfected Sf9 culture, uninfected Sf9 culture treated with DEB, and virus-infected Sf9 culture. These cultures are prepared the same way and at the same time as the mutagenized culture described herein.

The infected Sf9 insect culture is incubated at 27° C. for 1.5 hours.

### Chemical Mutagenesis

In a fume hood, DEB (other names 1,3-butadiene diepoxide or 1,2,3,4-Diepoxybutane) is added at a 0.1 mM final concentration to the culture. The culture is then incubated at 27° C. for 3 hours.

After the 3 hour incubation, the culture is moved to the fume hood. A cell scraper is used to detach cells from the 25 cm² flask. The cells and supernatant are moved to a 50 ml-conical tube and centrifuged at 900×g for 10 minutes at 4° C. The supernatant is removed to a specified waste container. Cells are washed with 10 ml PBS, incubated in fume hood for 5 minutes, then spun down at 900×g for 10 minutes at 4° C.

The wash supernatant is removed to a specified waste container. The cell pellet is then resuspended in 5 ml nudivirus media and moved to a new 25 cm² tissue culture flask, which is now considered DEB-free. Culture is incubated at 27° C. for 2 days.

After 2 days, the cell culture medium containing DEB-exposed HzNV-2 is collected after culture centrifugation at 900×g for 10 minutes and filter sterilization using a 0.22 μm filter. 2 ml of the virus stock is added to a new 25 cm² tissue culture flask containing Sf9 insect cells that were seeded at 1×10⁶ cells/ml in a 5 ml total volume with nudivirus media. The virus-infected culture is incubated at 27° C. for 7 d.

To purify and amplify the virus to a suitable volume for insect infection, the virus-containing medium was collected after centrifugation (3000 rpm, 10 minutes, 4° C.), filter sterilized using a 0.22 μm filter, and stored in 1 mL aliquots at −80° C. The titer of the virus is approximately 1×10⁴ pfu/mL While HzNV-2 has been shown to infect several lepidopteran cell lines including Sf-9 and TN-368 cells, Applicant found that it is difficult to pass the virus in insect cells due to the virus causing quick cellular lysis. The disclosed methods allow for amplification of the virus to a volume that allows for both amounts sufficient virus for storage and also virus suitable for insect infection.

Viruses were isolated using a traditional plaque assay, described in, for example, Anderson, D., Harris, R., Polayes, D., Ciccarone, V., Donahue, R., Gerard, G., and Jessee, J. (1996) Rapid Generation of Recombinant Baculoviruses and Expression of Foreign Genes Using the Bac-To-Bac® Baculovirus Expression System. Focus 17, 53-58. Large plaques, referred to as the p0 viruses, were further amplified.

For p0 to p1 amplification, Sf9 insect cells were seeded at 5×10⁵ cells/ml in a final 2 ml volume with nudivirus media in 12-well culture plates and incubated at 27° C. for 1 hour. Afterwards, viral plaques were picked using a large bore pipette tip and transferred to one well. Plates were incubated at 27° C. for 5 d. Medium containing DEB-treated HzNV-2 virus is collected after culture centrifugation (900×g for 10 m at 4° C.) and filter sterilized using a 0.22 μm filter.

For p1 to p2 amplification, Sf9 insect cells were seeded at 8×10⁵ cells/ml in a final 2 ml volume with nudivirus media in 6-well culture plates and incubated at 27° C. for 1 hour. Afterwards, 600 μl p1 virus was added to one well using a serological pipet. Plates were incubated at 27° C. for 4 days. Medium containing DEB-mutated HzNV-2 was then collected after culture centrifugation (900×g for 10 m at 4° C.).

For p2 to p3 amplification, Sf9 insect cells were seeded at 1×10⁶ cells/ml in 5 ml with nudivirus media in 25 cm² tissue culture flask with no incubation. ˜1.5 ml (or all) p2 virus was added to one flask using a serological pipet. Flasks were incubated at 27° C. for 4 days. Medium containing DEB-mutated HzNV-2 is then collected after culture centrifugation (900×g for 10 minutes at 4° C.) and filter sterilization using a 0.22 μm filter. p3 virus is stored in 1 ml aliquots at −80° C.

p3 virus was used to infect third instar larvae via the direct inoculation method. Each mutant virus caused an infection in the insect leading to formation of a viral plug found in agonadal female moths. Virus from these viral plugs may be used in subsequent experiments.

### Confirming Sterilizing Activity of Recombinant and Chemically Mutated HzNV-2 Virus

Taken together, the data demonstrate that recombinant and mutant HzNV-2 viruses can be effectively achieved using one or more of the above-described methods. Recombinant and mutant HzNV-2 viruses having a disrupted latency phase may be produced in which genes that affect the latency phase are disrupted or structural genetic elements required to establish or break latency are altered. The resulting genetically modified mutant HzNV-2 viruses cause elevated levels of sterility in infected H. zea moths. In one aspect, the genes that are disrupted include one or more of PAG1, ORF 90, and ORF92. It is noted that 100% sterile phenotypes may be produced by mutating different viral genes and regions of the viral genome.

TABLE 1
Locations of mutations in the
chemical mutants relative to HzNV-2.
Wild-type (WT) HzNV-2 virus was mutated
with DEB to form HzNV-2 chemical mutants.
Mutants KS-3, KS-45, and KS-51 were
sequenced and gene deletions were
identified. It is notable that the
three mutants have mutations in a region
that is not present in the HzNV-1 genome.

| HzNV-2 strain | WT base pairs affected | WT genes affected |
|---|---|---|
| KS-3 | 48 bp insertion at bp | ORF90, hypothetical protein |
| | 175,550 | |
| KS-45 | 80 bp insertion at bp | ORF90, hypothetical protein |
| | 175,650 | |
| KS-51 | 180,270-180,299 deletion | ORF92, hypothetical protein |
| | 77 bp insertion at bp | Intergenic DNA between |
| | 109,598 | hypothetical proteins |
| | | ORF55 and ORF56 |

The sterilizing activity of the resulting mutants were assessed through many experiments in which virus was injected into H. zea adults or third instar larvae. In a typical experiment, WT HzNV-2, the recombinant yfp HzNV-2, and mutant KS3 viruses were collected and purified from viral plugs found in virus-infected female moths and then injected into new healthy female moths 2 days after emergence. The eggs laid on oviposition day 3 were collected and the F1 progeny female adults were analyzed for sterility as indicated by the presence of a viral plug and number of eggs laid (Table 2). Both female groups infected with mutants yfp HzNV-2 and KS-3 laid few or no eggs and had a much higher percentage of agonadal females than the WT-infected group. Thus, the viruses obtained using the described methods are suited for use in sterilizing populations of pests susceptible to infection by the described viruses and may be utilized to control pest populations.

TABLE 2
Injection of female moths with mutant HzNV-2 KS3, recombinant
mutant yfp HzNV-2, or wild-type (WT) HzNV-2 and analyses
of their female offspring for sterility as determined by
the presence of a viral plug and production of eggs.

| Virus | Plugs | Eggs laid |
|---|---|---|
| WT HzNV-2 | 34% | many |
| KS3 | 95% | a few |
| yfp HzNV-2 | 100% | none |

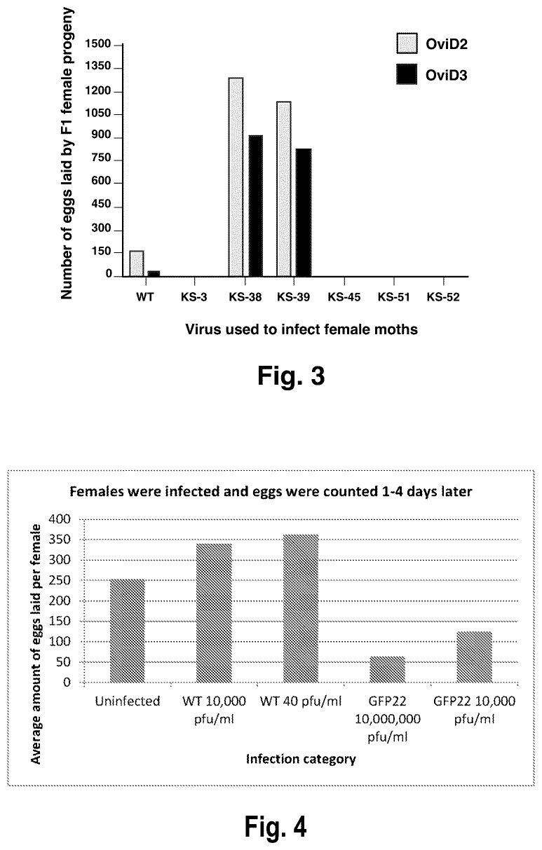

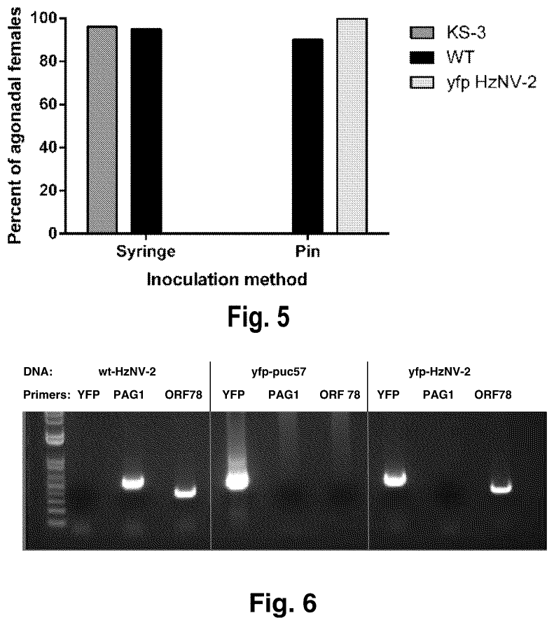

In a similar experiment, WT and 9 chemical mutant viruses were evaluated for the ability to cause agonadal moths. Briefly, adult female moths were injected with 100 μl of ˜10⁸ pfu/ml of virus isolated from viral plugs on the day of emergence. Eggs laid on oviposition days 2 and 3 were collected and reared to adult moths. The F1 progeny female moths were evaluated for the ability to lay eggs and the presence of a viral plug. Four mutants (KS-3, KS-45, KS-52, KS-51) caused viral plug formation in 100% of the F1 female progeny (FIG. 2). No eggs were laid by F1 female progeny of female moths infected with the three mutants (indicative of an agonadal phenotype (KS3, KS45, KS51) (FIG. 3). F1 female progeny of female moths infected with another mutant KS52, laid fewer eggs than F1 female progeny of female moths infected with WT virus on oviposition day 2 and no eggs on oviposition day 3.

The functional mutations defined by the applicant in ORFs 90 and 92 of HzNV-2 have several unrelated direct repeated sequences ranging from 24 to 81 bp in size and having these sequence repeated from 4 to 12 times. These repeated sequences were identified by Burand et al., (2012). Such repeated sequences may have structural as well as coding roles and with some functions of repeated sequences involving recognition sites for DNA-binding proteins and directing conformational changes of DNA that can promote DNA replication, DNA recombination and/or RNA transcription as examples. A similar repeated sequence exists in ORF 2 (direct repeat 1; Table 2; Burand et al., 2012) and is claimed herein as an identified sequence, region and ORF that is susceptible to mutation and such mutations are likely to impact DNA replication and recombination and the function of the virus relative to its effects on viral lysogeny and increased sterility among H. zea infected with mutations that alter ORF2 (SEQ ID NO: 3). It is notable that 3 mutations that increase sterility have been defined by the applicant and localize to ORFs 90 and 92 which contain 4 of the 6 repeated sequences in the HzNV-2 genome. ORF 2 and ORF 91 contain the only other large repeated sequences in the viral genome and are thus obvious candidates for mutagenesis with an expectation that it would impact HzNV-2 replication and lysogeny.

Table 2 shows additional data that compares genetically engineered and chemical mutant viruses. Progeny female moths developing from female moths infected with the genetically engineered recombinant virus, yfp HzNV-2 did not lay eggs and all F1 female progeny had viral plugs indicating their sterility. Similarly, essentially all F1 progeny of female moths infected with the chemical mutant viruses KS3, KS45, KS51, and KS52 had viral plugs and exhibited sterility (Table 2, FIGS. 2 and 3). By contrast, much smaller percentages (˜33%) of insects infected with WT virus were agonadal (had plugs) with most moths infected with the WT HzNV-2 being fertile and laying many eggs (Table 2, FIG. 3). Similar results are seen on other oviposition days, although the number of female sterile moths in the F1 progeny of female moths injected with WT HzNV-2 increases at later oviposition days as reported in the literature (Hamm et al., 1996; Burand 2013). These results support the hypothesis that inactivation of several nudivirus genes, for example, pag1 (SEQ ID NO: 6), ORF90 (SEQ ID NO: 4), and ORF92 (SEQ ID NO: 5) can cause sterility in essentially 100% of infected insects.

While it is to be recognized that viruses capable of being sexually transmitted among an insect pest species, can be mutated and selected using the described protocols without knowledge of specific genes involved in the phenotype of the resulting virus (for example, using the DEB protocol described above), some genes associated with increased sterility following viral infection have been identified by the Applicant. For example, the pag1, ORF 90, and ORF92 genes have been found by Applicant to be associated with increased sterility in virus-infected insects.

### Infection of an Insect Using Genetically Modified, Hypersterilizing Nudiviruses

Published nudivirus literature describes various techniques for infecting and sterilizing H. zea moths with WT HzNV-2 but are lacking in various aspects such that a meaningful protocol can be carried out. Applicant has developed protocols that efficiently 1) infect the adult moth, 2) infect the moth offspring, and 3) mimic natural infections of moth populations in the field.

Method of Sterilizing the Offspring of Adult Moths. A protocol for Producing Agonadal Infections with WT Sterilizing Nudiviruses.

To infect adult moths, the virus is injected into the abdomen of adult moths using an insulin syringe. In such experiments, WT virus collected from a viral plug effectively infected female adult moths and F1 progeny mimicking natural infected moth populations (i.e., 20-50% agonadal) (Table 3). Virus is collected by removing the plug from the abdominal area and suspending the plug in 100 μl PBS; this is called the unfiltered viral plug extract (UVPE). The virus is then diluted to 2% in 1× Grace's media and filtered through a 0.22 micron filter to sterilize; this is called the filtered viral plug extract (FVPE). Applicant determined there was no difference in the percentage of agonadal F1 progeny when using either UVPE or FVPE for injections. (Table 3.) Applicant used FVPE for most experimentation.

TABLE 3
Comparison of unfiltered viral plug extract (UVPE) and filtered viral
plug extract (FVPE) in their ability to cause agonadal F1 progeny with
plug. Oviposition day is the day the infected female moth laid eggs.
Eggs laid on oviposition days 3 and 4 were reared to adults.

| | | # Females | F1 progeny |
|---|---|---|---|
| Oviposition | Virus | evaluated | females |
| day | type | for plug | with plug |
| 3 | UVPE | 40 | 33% |
| 3 | FVPE | 25 | 36% |
| 4 | UVPE and FVPE | 35 | 74% |

### Developing the Protocol to Generate a High Number of Agonadal Moths

Subsequent experimentation determined that injecting virus extracted from viral plugs results in a higher percentage of agonadal F1 progeny than injecting virus produced from infections of Sf9 cells in culture. Briefly, adult female moths were injected with 50-60 μl of either FVPE or virus amplified in cell culture. Eggs were collected on oviposition day 3 and reared to adults. F1 female progeny were evaluated for the presence of a plug. Almost all of the F1 female progeny developed a plug if F0 female moths were injected with viral plug extract, whereas only 10% of F1 progeny developed a plug if injected with virus collected from infected Sf9 cells (Table 4).

TABLE 4
Comparison of virus isolated from infected Sf9 insect cells (cell culture)
and isolated from viral plugs (plug virus) in their ability to cause
agonadal F1 progeny as indicated by the presence of viral plugs.

| | | # Females | F1 female |
|---|---|---|---|
| Virus | Virus | evaluated | progeny |
| type | strain | for a plug | with plug |
| Cell culture | yfp HzNV-2 | 11 | 9% |
| Cell culture | KS-3 | 20 | 10% |
| Plug virus | yfp HzNV-2 | 26 | 100% |
| Plug virus | KS-3 | 20 | 95% |

Thus, the titer of virus amplified in cell culture used for infection of adult moths was not optimal. Titer is an important factor in developing a method for creating high volumes of sterile insects. The optimal viral titer will be as low as possible but sufficient to cause agonadal adults. To assess the effects of virus titer, adult female moths were injected with either 10⁷, 10⁴ or 10¹ pfu/ml virus (WT or yfp HzNV-2) and mated with uninfected males. Eggs were collected on oviposition days 3-5 and reared to the adult stage. Moths were evaluated for the presence of a viral plug and egg production. Virus titer of 10⁴ pfu/ml led to a high number of agonadal F1 progeny on oviposition day 5 (Table 5) indicating that the virus replication in the host moth was important for effective transmission of the virus to the offspring eggs.

TABLE 5
Moths were either uninfected or infected with a low (10¹ pfu/ml),
mid (10⁴ pfu/ml), or high (10⁷ pfu/ml) dose with either wild-
type (WT) or recombinant yfp HzNV-2 virus. Eggs were collected
on oviposition days 3-5, reared to adults, and the agonadal
state of each female was evaluated by the presence of a plug.

| | | F1 progeny | | | |
|---|---|---|---|---|---|
| | | collected on | | | |
| Virus | Virus | oviposition | # females | # Eggs | F1 females |
| group | dose | day | evaluated | laid | with plugs |
| Uninfected | — | Day 3 | 7 | ~300 | 0% |
| | | Day 4 | 6 | 14 | 0% |
| | | Day 5 | 4 | 0 | 0% |
| WT-infected | 10¹ pfu/ml | Day 3 | 9 | 5 | 0% |
| | | Day 4 | 5 | ~300 | 0% |
| | | Day 5 | 9 | 64 | 22% |
| | 10⁴ pfu/ml | Day 3 | 6 | 1 | 0% |
| | | Day 4 | 10 | ~150 | 20% |
| | | Day 5 | 10 | 0 | 100% |
| yfp HzNV- | 10⁴ pfu/ml | Day 3 | 4 | 0 | 0% |
| 2-infected | | | | | |
| | | Day 4 | 4 | 0 | 0% |
| | | Day 5 | 10 | 0 | 70% |
| | 10⁷ pfu/ml | Day 3 | 9 | ~150 | 0 |
| | | Day 4 | 3 | 0 | 0 |
| | | Day 5 | 1 | 0 | 0 |

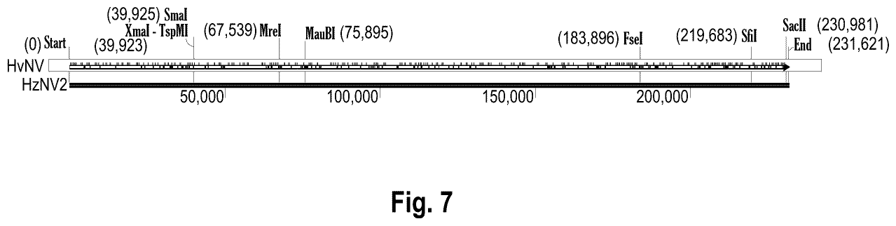

Injection of the recombinant virus into naïve male moths, followed by mating with healthy females, does not significantly reduce the number of eggs laid by female moths although the infection can be transmitted in this manner. However, when newly emerged female moths were injected with recombinant yfp HzNV-2 and mated with healthy males, the egg number was dramatically reduced (FIG. 4). This effect was dose dependent as a viral dose of 1×10⁶ pfu exhibited fewer eggs compared to a viral dose of 1×10³ pfu. The total number of eggs laid by female moths injected with WT HzNV-2 was not reduced relative to media injected controls; this effect is only observed with the mutant and recombinant viruses.

### Methods of Sterilizing Adult Moths by Injecting the Insects with Virus at the Larvae Stage

An alternative protocol for inducing sterility is to inject virus into third instar larvae such that the injected insects exhibit the sterile pathology as adults. While this is not the normal mode of transmission, larval injections are much faster, amenable to automation and likely mimic the activation of viral replication (Rallis et al., 2002-a). Applicant has induced sterility in third instar larvae by syringe injection and direct inoculation with a needle dipped into a virus solution containing 10⁶ pfu/ml.

### Infection Using Syringe Injection

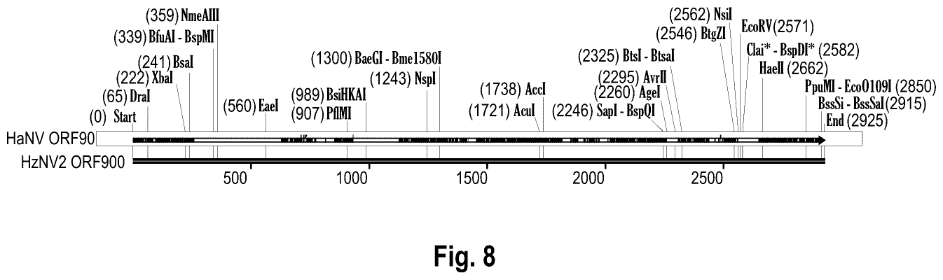

Injecting supernatants from cultures of virus-infected cells into adult moths does not produce high numbers of agonadal offspring (Table 4). To determine if virus produced from infected, cultured insect cells effectively initiated productive virus infections when injected into larvae, Applicant injected virus-containing tissue culture medium into third instar larvae. third instar larvae (83 larvae per group) were injected using an insulin syringe with either WT HzNV-2, yfp HzNV-2, or KS-3 virus (25-50 μl) or not injected. Larvae were reared to adults. Surprisingly, none of the yfp HzNV-2-injected larvae pupated and eventually died (Table 6). However, ˜95% of female larvae infected with either WT HzNV-2 or KS-3 virus produced a viral plug as adults (FIG. 5). Applicant concluded that while injecting virus amplified in tissue culture into adult moths does not produce many agonadal offspring, injecting virus amplified in tissue culture into third instar larvae was an effective method as almost all moths became agonadal.

TABLE 6
Larval and pupal stage mortality after injection of
third instar larvae with WT HzNV-2, recombinant and mutant
HzNV-2 derived from virus-infected insect cell cultures.

| | Insects survived |
|---|---|
| Group | injection and pupated |
| Uninfected | 100% |
| WT-infected | 60% |
| KS3-infected | 73% |
| yfp HzNV-2-infected | 0% |

Injection of viral plug extract into third instar larvae with an insulin syringe was also evaluated. Briefly, WT HzNV-2 and yfp HzNV-2 FVPE was diluted to 7×10³ pfu/ml and 2.5×10² pfu/ml and 25 μl was injected into third instar larvae. Larvae were reared to adults and female moths were evaluated for the presence of a plug. Female moths that were injected as larvae with a titer of 10³ pfu/ml developed a viral plug, whereas only ˜80% of those injected with 10² pfu/ml developed a viral plug (Table 7). Applicant concluded that injecting third instar larvae with viral plug extract was also highly effective at developing agonadal adults, but titer should be around 10³ pfu/ml.

TABLE 7
Incidence of viral plugs after third instar larvae were
infected with wild-type (WT) HzNV-2 or recombinant yfp HzNV-2\at viral
doses between 10² to 8 × 10³ pfu/ml.

| Infection- | Virus titer | Females |
|---|---|---|
| group | (pfu/ml) | with plug |
| Uninfected | 0 | 0% |
| WT-infected | 1 × 10² | 80% |
| WT-infected | 1 × 10³ | 100% |
| yfp-HzNV2- | 4 × 10² | 79% |
| infected | | |
| yfp- HzNV2- | 8 × 10³ | 100% |
| infected | | |

### Infection Using Direct Inoculation Using a Pin

Direct injection of third instar larvae or moths are efficient methods for infecting HzNV-2 in the laboratory, but feeding is a preferred method.

Infecting H. zea with WT HzNV-2 via an oral route of infection is possible. Raina et al. (2006) fed WT HzNV-2 to 1^{st} instar larvae for 1-3 days and found that 9-17% (varies based on gender and duration of feeding) of adults became agonadal. Hamm et al. (1996) fed WT HzNV-2 to adult moths in an aqueous solution and from 60-100% of the offspring adults were agonadal. The HzNV-2 genome also encodes genes related to four baculovirus genes (p74, pif-1, pif-2, and pif-3) whose protein products are involved in viral entry per os (Burand, Kim, Afonso et al. 2012). Although the natural route of infection for HzNV-2 is through mating and/or transovarial transmission, other methods for infecting insects include direct inoculation and feeding of both larvae and adults.

A direct inoculation method for transmitting the virus to third instar larvae (Table 8) was developed and may be amenable to automation. This method is similar to that used by Hamm et al., (1996) to infect 1^{st} instar larvae with viral plug extract with 9 of 10 larvae becoming agonadal as adults. Direct inoculation is a rapid means to introduce virus into H. zea larvae in which a sterile pin is dipped into the viral solution and then used to prick larvae between the head capsule and abdomen with sufficient force to penetrate the cuticle and enter the insect's body cavity. WT HzNV-2 (A, B) virus obtained from two different cell culture infections A and B) were used for direct inoculation. The pricked larvae were reared to adult moths, and female moths were evaluated for the presence of a plug. It was observed that 57% (WT A) and 16% (WT B) of moths were agonadal.

To test virus isolated from plugs, ˜10⁸ pfu/ml of WT HzNV-2 or recombinant yfp HzNV-2 viruses were isolated, filter sterilized and used in direct inoculation experiments. The inoculated larvae were reared to adult moths and mated. All female moths developing from larvae inoculated with recombinant yfp HzNV-2 were sterile (no eggs laid; plugs in 100% of females). Upon dissection, all male moths examined were found to be agonadal.

For larvae inoculated with WT HzNV-2, 90% of female moths had a viral plug with the number of eggs laid commensurately reduced relative to controls (Table 8). In summary, the direct inoculation method was not only as efficient as injecting the virus into larvae, it was also much faster.

TABLE 8
Effects of direct inoculation of third instar larvae
with wild-type (WT) and yfp recombinant HzNV-2
purified from viral plugs on moth sterility.

| | | | State of |
|---|---|---|---|
| | # of | % Female | reproductive |
| | eggs | moths with | organs in male |
| Group | laid* | plugs | moths** |
| Unpricked | High | 0 | 0% agonadal |
| Medium control | High | 0 | 0% agonadal |
| WT HzNV-2 | Low | 90 | 50% agonadal |
| yfp HzNV-2 | None | 100 | 100% agonadal |

*high: >700 eggs; low <200 eggs;
**determined by dissecting reproductive organs of H. zea males.

To investigate effects of titer on the direct inoculation method WT HzNV-2 isolated from a viral plug was diluted to 10⁶ pfu/ml and used to inoculate third instar larvae. Only 18% of WT HzNV-2 pricked females were agonadal with the 10⁶ pfu/ml inoculum. Many females, termed carriers, are infected with virus but have inactive or latent infections. Moths having latent infections have intact, functional reproductive tracts but can transmit the virus horizontally and vertically to their offspring. PCR using a primer set to the HzNV-2 ORF78 was performed, and all 20 of the females without viral plugs tested were carriers. Applicant concluded that use of direct inoculation with a lower titer of 10⁶ pfu/ml to infect third instar larvae created a carrier population.

### Method of Infecting an Insect with the Disclosed Viruses

Alternative methods for introducing insect nudiviruses and baculoviruses are reported in the scientific and patent literature. The literature suggests that efficient infection is possible by feeding newly emerged (neonate) first instar larvae, by feeding adults virus in a sucrose solution, by adding fluorescent brighteners to larval diet, or by aerosol infections with virus in powdered or droplet form (Kirkpatrick et al., 1994; U.S. Pat. No. 7,261,886). A recent patent filing reports efficient baculovirus infection achieved by immersing larvae in a viral solution (US Patent Publication US2011/0314562).

In accordance with the instant disclosure, three different methods for infecting large numbers of larvae may be used as follows:
1. Virus Feeding. The protocol used by Raina and Lupiani (2006) may be used. Briefly, newly hatched H. zea larvae may be placed in a 100×15 mm Petri dish containing a diet with 1000 pfu of mutant HzNV-2. To increase the efficient uptake of the virus, the fluorescent brightener Blankophor may be added to the diet (Martinez et al., 2009). The larvae may be allowed to feed for 48 hours then placed in diet-containing cups. H. zea is very cannibalistic and so must be housed individually at a young instar. The pupae will be sexed and emerged females will be analyzed for viral plugs, the ability to lay fertile eggs after mating, and the presence of intact reproductive organs.

The males may then be dissected and their reproductive organs examined to determine if they are agonadal. The presence of mutant HzNV-2 may be confirmed by PCR, as described above.
2. Virus Aerosols. The virus may be delivered as a lyophilized powder (Kirkpatrick et al., 1994) or as an aqueous mist (U.S. Pat. No. 7,261,886). third instar H. zea larvae may be anesthetized with CO₂ and placed in a test chamber. The lyophilized mutant virus may be placed in the chamber at different doses and dispersed continuously throughout the chamber by a gentle stream of air, for a total exposure time of 30 minutes. Each insect may be sexed after they pupate, and after emergence, each moth may be analyzed for sterility as described above. For the aqueous mist deliver, a Potter precision laboratory spray tower (Burkard Scientific) may be used, and third instar larvae may receive doses of mutant HzNV-2 from 10² to 10⁴ pfu/ml. Agonadal pathology may be assessed in adult moths.
3. Immersion. H. zea larvae may be submerged in a HzNV-2 solution as described by Lu et al. (2011). This treatment method involves first stressing the insects at 4° C. for 15 hours before soaking the third instar larvae in different concentrations of mutant HzNV-2 (10³-10⁶ pfu/ml) for 1 hour. Sterility in adult moths may be determined as described above.

### Methods for Producing Diapausing Pupae from Virus-Infected Moths

In view of the contemplated aspects for producing moths, it may be necessary, due to a variety of factors or circumstances, to delay moth emergence. To achieve such a delay, the following method contemplates a follow on procedure to other instantly disclosed moth producing aspects where conditions require a delay in production of moths.

To produce diapausing, virus-infected Helicoverpa zea pupae, adult female moths are injected 24 hours post-eclosion with 10 μL of a viral suspension containing 1×10⁶ viral particles in PBS (pH 6.8). Injections are performed in the lower third of the abdomen using standard microinjection techniques.

Injected females are then placed with uninfected males at a 1:1 ratio and allowed to mate under standard laboratory conditions (14:10 Light: Dark cycle, 30° C.).

Egg collection begins at 48 hours post-mating (first night of eggs are discarded). Eggs are placed into PLA grids with corn earworm diet, and clear lids to allow for light penetration.

Larvae are reared in these grids at a 14:10 Light:Dark (L:D) cycle at 30° C. until they reach the third instar stage.

At the third instar stage of development, the entire grid is transferred to an incubator or rearing room set to 17.5° C. with a short-day photoperiod of 8:16 L:D, which induces diapause.

Once larvae pupate, pupae are either moved to an empty grid, or moved to individual empty cups, both maintained with clear lids. Diapause is confirmed at the pupal stage based on morphological characteristics, specifically, the continuing presence of larval eye spots on the pupal case.

Pupae are maintained in diapause for up to 2 months, though longer diapause durations may be possible, and additional testing is ongoing to determine the upper limits of pupal viability and responsiveness to termination cues.

To terminate diapause, pupae are transferred to a rearing room maintained at 30° C. and a 14:10 L:D photoperiod, which promotes continuation of development and adult emergence.

Upon emergence, infection status can be verified through visual morphology (agonadal status/viral plug presence in females) or molecular detection (PCR).

### Method of Infecting a Target Insect Through Delivery of the Disclosed Viruses Within or on Target Pest Eggs

A new approach to introduce pathogens to target insect pest populations by delivering the pathogen within or on pest eggs is further explained below.

An aspect of the new method includes laboratory rearing of egg deliverable materials, where delivery of Helicoverpa zea or Helicoverpa armigera eggs to plants is reliant on efficient methods of production of these insects and efficient methods for infecting the adult female moths which produce the eggs. The focus of producing such eggs includes methods required to collect the active ingredient, the H. zea and armigera eggs infected with the active ingredient HzNV2 isolate 90DR71 or similar viruses with genes ORF 90, ORF 92 or PAG1 inactivated by genetic mutation, CRISPR modification or recombinant DNA methodology.

In this aspect of the invention with regard to lab rearing, when female moths of these species lay their eggs, they are covered with a biological secretion from oviduct glands that adheres the egg to plant surfaces in the field or to artificial oviposition substrates in the laboratory. The laboratory oviposition substrate consists of a fiberglass screen mesh (or other similar laboratory substrate materials including nylon or polyethylene mesh, stainless steel mesh, woven polyester fabric, or 3D-printed grids composed of materials such as poly lactic acid, PLA) upon which moths hang in an inverted position and a plastic substrate consisting of poly lactic acid (PLA); or other suitable plastic substrates such as acrylonitrile butadiene styrene (ABS), acrylonitrile styrene acrylate (ASA), polyethylene terephthalate (PET), glycolyzed polyester (PETG), polycarbonate (PC), polyether ether ketone (PEEK), polyether ketone ketone (PEKK), polypropylene (PP), or blends or composites thereof. The substrate may also include fiber-reinforced variants or hybrids with natural or organic materials, such as corn byproducts, cellulose, bamboo, cork, or wood. These substrates may be further modified with surface coatings or treatments, such as silicone spray or polytetrafluoroethylene (PTFE) to reduce adhesion and facilitate egg removal. The key criteria for selection include compatibility with the moth's oviposition behavior and the ability to allow gentle removal of eggs without physical damage or clumping.

These materials minimize the adhesive properties of the oviduct secretion such that eggs can be removed by minor physical force (e.g., brushing the eggs off with a facial brush or by vigorous agitation such as a 1-minute shaking with a Vortex laboratory agitator). The essential factor for these materials within this aspect of the invention is that the oviduct secretion forms a poor surface bond with the substrate for proper egg mobilization.

Many materials having similar physical characteristics are contemplated to be used with the selection criteria simply being a non-adhesive surface. By contrast, fibrous, natural materials are not suitable because the eggs cannot be removed from these substrates without damage.

In furtherance of the above aspect of the invention, in a preferred embodiment, the eggs can be removed without wetting their surfaces because this facilitates their subsequent distribution.

There are aqueous solutions, such as a dilute (5%) bleach solution followed by plain water, that can dissolve the adherent oviduct solution, however these solutions produce eggs that readily adhere to each other and form clumps that are poorly suited for distribution in the absence of a surfactant.

An embodiment of the egg application method is to deliver dry eggs to damp plant surfaces. Such an application would provide for eggs to be wetted by the moisture on plant surfaces which reactivates the adhesive properties inherent in the oviduct secretion that coat the egg surfaces.

This application embodiment is best accomplished manually in small plots or with a mechanical distribution method such as aerial drones for larger acreages. In a preferred embodiment, the eggs are mixed with an inert granular substrate such as sand to increase the volume of material and thereby support effective distribution. Many inert materials that are suitable for effective distribution are contemplated (e.g., cornstarch, flour, ground rice hulls, vermiculite, cellulose powder, or finely milled agricultural byproducts such as wheat bran or oat husks). These materials may be selected based on factors such as particle size, density, flowability, electrostatic properties, and biological compatibility with the eggs and plant surface. The ideal substrate supports uniform dispersal, prevents egg clumping, and is non-toxic to both the insect eggs and the plants.

Another embodiment of the application method is to dilute the eggs in a large volume of aqueous solution in a conventional sprayer. The eggs utilized are approximately 0.5 mm in diameter and have a weight of 1-2 micrograms each. The utilized conventional sprayer nozzle aperture must be at least 25% larger than the egg diameter (or 0.625 mm), and up to the aperture size that allows for misting of the plant, rather than drenching the plant which would wash eggs off plant surfaces. For this embodiment, the spray nozzle size optimal for misting is 1.25 mm.

In another embodiment, the method contemplates egg application density and/or dosage. The density of eggs being applied to a plant material can vary depending on the intended purpose of the application. At the lowest application rates, the embodiment contemplates that the application rate produces 1 mature adult moth per 25 plants. It is known that predation of insect eggs has been measured at 95% (Vargas, Roger & Nishida 1980). In view of this, the embodiment of a low-density approach involves applying 1 egg per plant, or about 20,000 eggs/acre in sweet corn.

In a further embodiment, a high-density application is contemplated that the treatment objective is to yield one adult moth per plant, as many as twenty eggs may be applied per plant, resulting in as many as 400,000 eggs per acre. The high-dose treatment of this embodiment is particularly suitable for smaller treated acreage within a much larger untreated area. This treatment would be appropriate for non-BT transgenic ‘refuge’ within a predominantly insect-resistant BT transgenic crop. In this situation, the refuge becomes a production center for sterile Insterus-Hz moths capable of suppressing BT resistance in the field.

An alternative embodiment application targets delivery of Insterus-Hz moths at the level of 10-20% of the feral population. This 10% threshold has been shown to be sufficient in laboratory, field and model systems to infect over 90% of the uninfected, feral moths in a population.

In another embodiment, the method contemplates egg handling, where the viability and integrity of insect eggs during storage, transport, and application is dependent on careful control of environmental conditions. Two major concerns of egg handling are desiccation of the eggs in overly dry conditions, and conversely, the tendency of eggs to clump together in the presence of excess moisture, which can interfere with accurate dosing and delivery.

In the embodiment, once eggs hatch, larvae produce silk and moisture that can exacerbate clumping. In order to prevent premature hatching, eggs should be stored at cool temperatures. Refrigeration in a standard 4° C. refrigerator suppresses egg development without causing injury from cold. However, refrigeration can result in moisture condensation on egg surfaces, particularly during temperature fluctuations or in humid storage conditions.

In the above embodiment, in order to address moisture, a mild desiccant is used within the refrigerated environment to absorb ambient moisture and keep the area around the eggs relatively dry. Importantly, the desiccant must not come into direct contact with the eggs to avoid excessive dehydration.

A variety of mild desiccants are contemplated in the above embodiments which include but not limited to rice husks, rice powder, food-grade silica gel packs, vermiculite, or corn starch.

In the above embodiments, eggs stored in the above conditions remain and do not adhere to the substrate or each other, and are suitable for downstream application. Eggs should at no point be wetted during handling, as water exposure causes the eggs to adhere into clumps, which are not suitable for field application. The goal is to preserve the dry, non-adhesive state of the eggs until the point of application, without lethal levels of desiccation for the eggs.

In another embodiment, the method contemplates the application of eggs to plants with the use of powder. In this embodiment insect eggs may be applied to plant surfaces in a dry (powder) suspension formulation. The powder coated form offers advantages in terms of case of handling, shelf-stability, and compatibility with aerial or ground based mechanical dispersal equipment.

For powdered applications, it is critical that the eggs remain free-flowing, where the eggs do not adhere to one another or to equipment, where such adherence would interfere with accurate dosage. To achieve such free-flowing eggs within the embodiment, the eggs are combined with anti-clumping agents. These agents are inert, non-toxic powders that act as physical separators between the eggs. Suitable materials for this purpose include but are not limited to cornstarch, flour, vermiculite, ground rice hulls, cellulose powder, or finely milled agricultural byproducts such as wheat bran or oat husks.

In a further embodiment, when visualization of the egg distribution is needed, a fluorescent dye can be used for physical separation, and can be incorporated into the powder.

For the above embodiment, all components of the powder formulation must be non-toxic to the insect eggs, non-toxic and not damaging to crop plants, physically compatible with dispersal equipment, and effective at preventing egg clumping.

Dispersal of powdered eggs requires equipment that does not crush the eggs, and allows them to flow through the equipment and onto the plants. The aperture size of the dispersal device must be at least 1.5 mm to allow individual eggs to pass through. Depending on the formulation and powders used, larger pore sizes may be necessary to accommodate the coating on the eggs.

In the above embodiment, various modes of powder formulation for egg dispersal can be employed. Such modes of powder formulation for egg dispersal can be via bulk release, by batch load, or mechanized ground dispersers and by drone.

In the above embodiment, bulk release of eggs is performed through gravity fed or vibrating nozzles/drop ports. Alternatively, release of eggs is performed through batch-loaded chambers that release a defined number of eggs at controlled intervals. Alternatively, release of eggs is drone based or by mechanized ground dispersers.

The powder application format for the above embodiment is particularly suited to early morning applications when dew may aid in egg adhesion.

For another embodiment, liquid applications are contemplated, where insect eggs may be applied to plant surfaces in a liquid suspension formulation. The liquid suspension method offers compatibility with conventional agricultural spray equipment already in widespread use. This approach has the advantage of integration into existing crop treatment plans with minimal equipment modification. This method is particularly useful for large-scale applications or when environmental conditions, such as dry foliage, reduce the efficacy of powdered dispersal.

The carrier solution used in liquid applications must be carefully selected to support egg viability during mixing, storage, and spraying. Acceptable carriers include but are not limited to saline, water, or buffered solutions, provided that the pH and salinity are within an acceptable range to avoid osmotic or chemical damage to the eggs.

Maintaining a homogenous suspension in the above embodiment is essential for consistent dosing during application. Because insect eggs eventually settle out of solution, the formulation should be agitated continuously or intermittently using a method such as mechanical agitation (rotating paddles, stir bars), jet agitation (recirculating sprays), or manual agitation (shaking or swirling in handheld tanks). Such agitation must be gentle enough to avoid mechanical damage to eggs, but be sufficient to prevent sedimentation.

In the above embodiment, several classes of additives may be included in the liquid formulation to improve dispersion, suspension, and adherence of eggs to plant surfaces. Such additives include surfactants, suspending agents and wetting agents.

Surfactants reduce surface tension to reduce egg aggregation. Exemplification of surfactants include, but are not limited to, plant-safe dish soaps, polysorbates (polysorbate 20 or polysorbate 80), and other agricultural adjuvants formulated for foliar application. Suspending agents increase solution viscosity to minimize settling of eggs. Exemplification of suspending agents include, but are not limited to xanthan gum, guar gum, carboxymethyl cellulose (CMC), sodium alginate (SA), or methylcellulose. Wetting agents enhance spreading of droplets across plant surfaces, increasing contact and adhesion. Exemplification of a wetting agent includes, but are not limited to, polyalkyleneoxide modified heptamethyltrisiloxane.

For liquid applications, nozzle selection must balance flow rate, misting quality and droplet size to ensure eggs are not damaged or clogged within the equipment. Additionally, aperture sizes should be at least 25% larger than the egg diameter, and spray patterns should favor leaf surface coverage, rather than run off or drenching. Liquid applications are particularly suited to times when the leaf surface would be dry, inhibiting adhesion of the powder formulation.

For another embodiment, egg application timing is critical to success of field deployment, affecting adhesion of the eggs to the plant surface and the survival of the eggs in the environment. Selection of the appropriate application timing window should be informed by environmental conditions including temperature, humidity, dew point, and wind speed.

In view of the above embodiments, morning applications are ideal for powdered formulations. During this time, dew formation provides surface moisture on leaves, reactivating the oviduct secretions coating the egg. This improves adhesion of the dry eggs and powder to the plant surface and assists eggs in falling into crevices such as whorls, where they may be more likely to be protected from environmental stress and predation.

In view of the above embodiments, afternoon or evening applications are generally better suited for liquid formulations, especially in dry climates or during peak heat. When applied under drier conditions, the liquid suspension ensures eggs adhere to the plant surface before evaporation occurs, and before desiccation of the eggs occurs in high midday temperatures.

In view of the above embodiments, environmental considerations are essential for viability and effective distribution of eggs. Egg application of the above embodiments should be avoided under conditions of high wind, high heat, and rain. High winds can result in uneven deposition. High heat can cause rapid desiccation of eggs, while rain may wash eggs off treated surfaces.

Monitoring of humidity, temperature, and wind conditions is recommended to guide application timing.

For the above embodiments, ideal conditions include moderate humidity, moderate temperatures, low wind, and low chances of rain for the following 24 hours. This will enhance egg adherence and survival post application.

A further embodiment involves egg application strategies, where effective deployment of eggs must consider the biology of the moth, as well as spatial dynamics of the crops. The goal of application is not necessarily to achieve even egg distribution across every plant, but rather to strategically place eggs in a way that maximizes egg survival and larval development to the adult moth. Another goal of the embodiment is to optimize timing of adult eclosion and subsequent viral transmission to the target pest population, which takes advantage of the highly mobile nature of adult moths.

Another embodiment regarding egg application strategies involve the use of refuge plots that are designated areas within or adjacent to crops where infected insects can develop. These refuge zones would include no Bt traits or insecticidal sprays. These untreated zones would provide safe habitat for the Insterus-Hz infected eggs to develop into adult moths. These moths would then disperse naturally into surrounding treated areas, transmitting the virus. This method does not require larvae to develop on the target crop, which may be important in crops where damage would be considered problematic.

Another embodiment regarding egg application strategies involves treating alternating rows within a field. Such strategies include, but is not limited to, applying infected eggs to every 10^{th} crop row, which may be sufficient to establish a source population of infected moths that will mate with the pest population, and effectively transmitting the virus without requiring blanket coverage. Such a strategy approach reduces input costs and labor while maintaining effectiveness.

Another embodiment regarding egg application strategies is to seed corn production, where “male” and “female” plants are planted in separate crop rows, targeting the male rows for egg application provides a focused, efficient strategy. Since male plants are not harvested and serve to produce pollen, applying infected eggs to these rows ensure moths develop on part of the field that is not the primary economic part, minimizing risk to yield while still transmitting the virus.

### Method of Protecting a Crop from Pest Insects

The instant disclosure addresses a method for control of lepidopteran pest moths by rendering them sterile from infection with mutant or transgenic sexually transmitted nudiviruses. The delivery of HzNV-2 or mutants formed thereof in accordance with the disclosed methods and compositions to the targeted population may be through established methods for release of moths for sterile insect control. In one aspect, the moths or other pest infected with a mutant virus as disclosed herein, are released at point locations and permitted to disperse over a range. The range may be, for example, about 800 meters from the release site or released aerially from planes, helicopters or drones. Moths infected with mutant or recombinant HzNV-2 may be released after infection using one or more of the disclosed methods at ratios from 0.1 infected moths/WT moth in the field population moth up to 10 infected moths/WT moth in the field population. Targeted release at lower ratios may rely on generational transmission of the infection for control and may require supplemental release on virus-infected adult moths. Similarly, the delivery of HvNV, HaNV or mutants formed thereof in accordance with the disclosed methods and compositions to the targeted population described herein, may be used through established methods for release of moths for sterile insect control. We have, therefore, identified and isolated the genomic sequences of HvNV (SEQ ID NO: 20); the predicted peptide sequence of HvNV ORF 90 (SEQ ID NO: 21); the predicted peptide sequence of HvNV ORF 92 (SEQ ID NO: 22) and the nucleotide sequence of HvNV pag1 (SEQ ID NO: 26). We have, also identified and isolated the genomic sequences of HaNV (SEQ ID NO: 23); the predicted peptide sequence of HaNV ORF 90 (SEQ ID NO: 24); the predicted peptide sequence of HaNV ORF 92 (SEQ ID NO: 25) and the nucleotide sequence of HaNV pag1 (SEQ ID NO: 27).

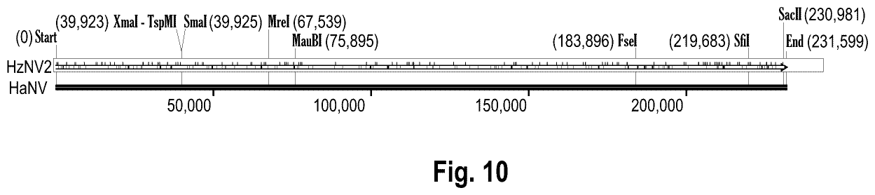

FIG. 7 is a schematic alignment of HvNV with HzNV-2 genome. This shows a high level of conservation of sequence (over 80%), predicted peptide sequence and gene order (synteny) within the viral genome. Such a high level of conservation is prima facia evidence of a required and conserved genetic function making it predictable in the state of the art to target the conserved genes to reproduce the effects mutation known to enhance sterility in the HzNV-2 genome. This it is predictable that mutations in HvNV and HaNV ORF 90, ORF 92 and pag1 will increase sterility in these viruses as they are known to do with the HzNV-2 genome.

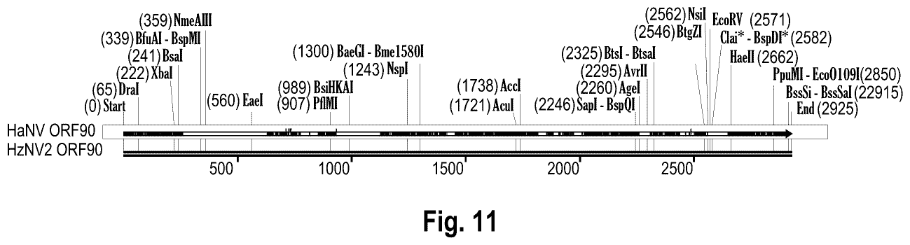

FIG. 8 is an alignment of HvNV ORF 90 with HzNV-2 ORF 90. This shows conservation of nucleotide and predicted amino acid sequences that is consistent with conserved functionality of ORF 90 between these viruses.

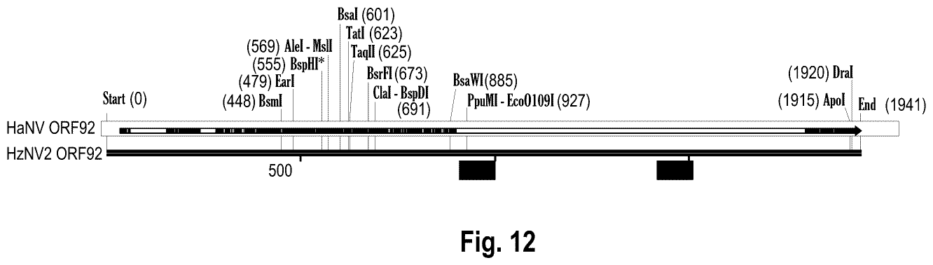

FIG. 9 is an alignment of HvNV ORF 92 with HzNV-2 ORF 92. This shows conservation of nucleotide and predicted amino acid sequences that is consistent with conserved functionality of ORF 92 between these viruses.

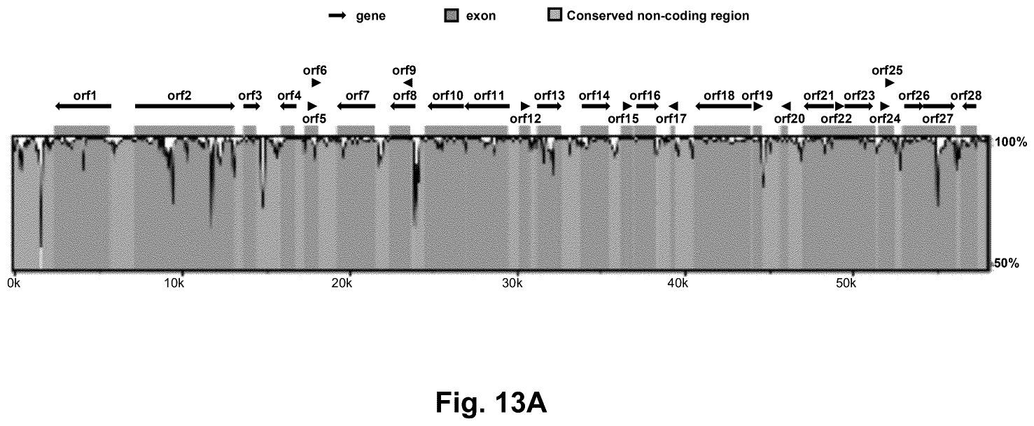

FIG. 10 is a schematic alignment of HaNV with HzNV-2 genome. This shows a high level of conservation of sequence (over 80%), predicted peptide sequence and gene order (synteny) within the viral genome. Such a high level of conservation is prima facia evidence of a required and conserved genetic function making it predictable in the state of the art to target the conserved genes to reproduce the effects mutation known to enhance sterility in the HaNV-2 genome. This it is predictable that mutations in HaNV and HaNV ORF 90, ORF 92 and pag1 will increase sterility in these viruses as they are known to do with the HzNV-2 genome.

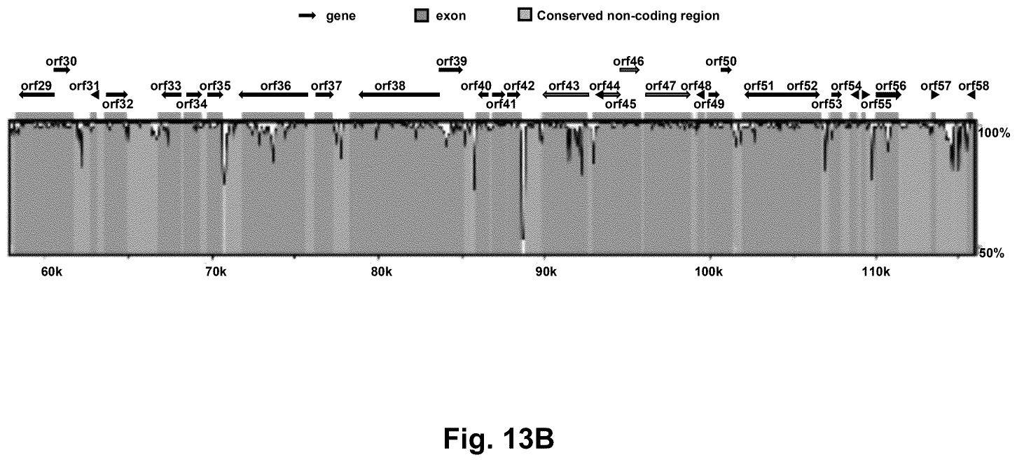

FIG. 11 is an alignment of HaNV ORF 90 with HzNV-2 ORF 90. This shows conservation of nucleotide and predicted amino acid sequences that is consistent with conserved functionality of ORF 90 between these viruses.

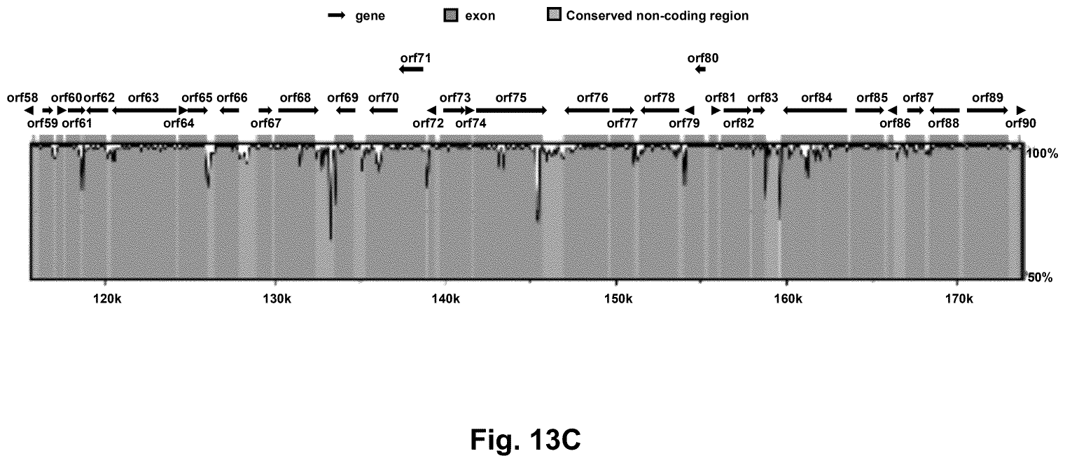

FIG. 12 is an alignment of HaNV ORF 92 with HzNV-2 ORF 92. This shows conservation of nucleotide and predicted amino acid sequences that is consistent with conserved functionality of ORF 92 between these viruses.

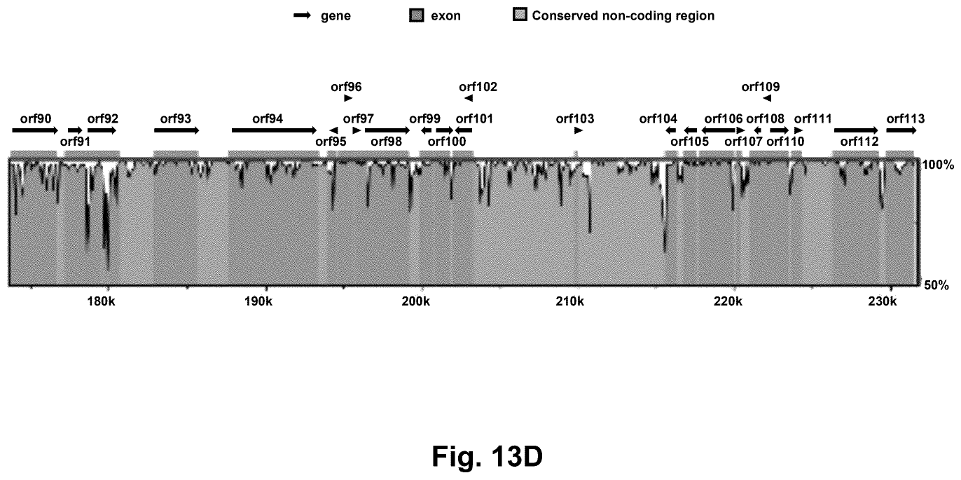

FIG. 13A-13D, together show the whole genome alignment of HvNV main contig and HzNV-2 genome. The bottom line represents the HzNV-2 genome and the top line represents the HvNV genome. Nucleotide variations are indicated as vertical lines and gaps. ORF distribution of HzNV-2 genome shown on top and the sequence similarity is represented at 100 bp resolution ranging from 50% to 100%. This shows sequence conservation and synteny across the entire genome consistent with viruses having closely related biological functions and pathology but preferential infect different species and are expected to be infectious to an overlapping host range when infected by sexual transmission, injection or feeding.

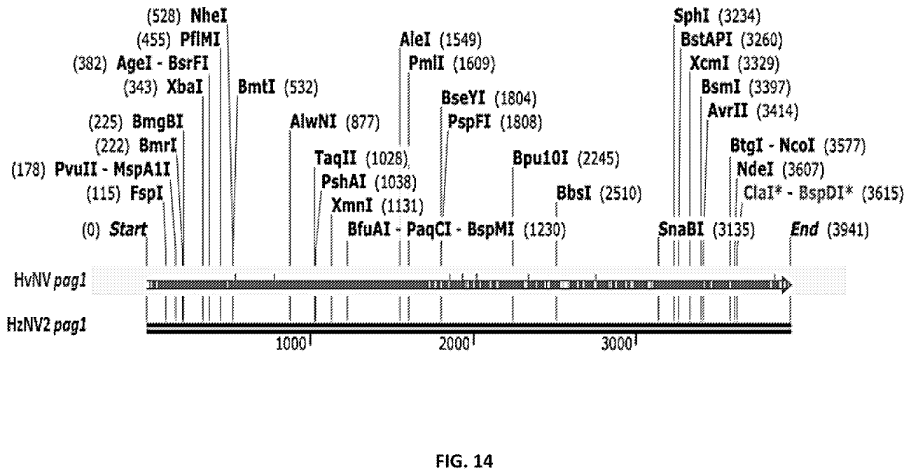

FIG. 14 is a schematic alignment of HvNV pag1 and HzNV-2 pag1 genome. This shows sequence conservation of this non-coding gene known to be involved in latency and known to increase sterility when mutated in HzNV-2.

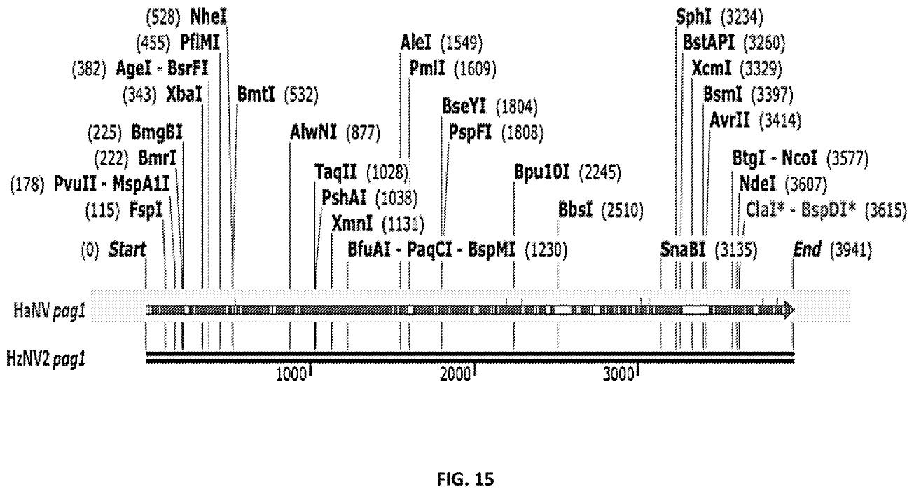

FIG. 15 is a schematic alignment of HaNV pag1 and HzNV-2 pag1 genome. This shows sequence conservation of this non-coding gene known to be involved in latency and known to increase sterility when mutated in HzNV-2.

Taken together, the evidence above supports genetic modification of the HvNV and HaNV nudiviruses in the identical manner as the HzNV-2 nudivirus and that the genetically modified HvNV and HaNV nudiviruses would be capable of being sexually transmitted by an insect useful for controlling pest populations. The genetically modified HvNV and HaNV nudiviruses would be capable of causing sterility in a target population of insects to control an insect pest population and reduce the crop damage caused by the target population of insects.

An implementation of the present invention may comprise a lepidopteran insect infected with a virus comprising a genetically modified Heliothis virescens nudivirus (HvNV) having at least one mutation in a region selected from the group comprising HvNV Open Reading Frame 90 (HvNV ORF90) (SEQ ID NO: 21), HvNV Open Reading Frame 92 (HvNV ORF92) (SEQ ID NO: 22) and HvNV Persistence-associated Gene (HvNV pag1) (SEQ ID NO: 26) to disrupt expression of the gene product encoded by said mutant region, wherein said virus causes increased lepidopteran insect sterility as compared to a wild-type HvNV virus.

The lepidopteran insect can be selected from the group consisting of Helicoverpa zea (H. zea) H. armigera, H. assulta, Heliothis virescens, Agrotis ipsilon, Spodoptera frugiperda, and Spodoptera exiguae.

An implementation of the present invention may comprise a method of making a lepidopteran insect capable of transmitting a virus comprising a genetically modified Heliothis virescens nudivirus (HvNV) having at least one mutation in a region selected from the group comprising HvNV Open Reading Frame 90 (HvNV ORF90) (SEQ ID NO:21), HvNV Open Reading Frame 92 (HvNV ORF92) (SEQ ID NO: 22), and HvNV Persistence-associated Gene (HvNV pag1) (HvNV ORF92) (SEQ ID NO: 26), to disrupt expression of the gene product encoded by said mutant region, wherein said virus causes increased lepidopteran insect sterility in said lepidopteran insect, comprising infecting said lepidopteran insect with said virus.

The virus can be derived from a viral plug.

The virus can be administered orally to said lepidopteran insect.

The virus can be administered to said lepidopteran insect via direct inoculation of insect larvae or adult lepidopteran insect by puncturing the cuticle with a pin containing viral inoculum derived from a viral plug.

The virus can be administered to said insect via direct hypodermic injection into third instar larvae or moths.

An implementation of the present invention may comprise a method of protecting a crop from lepidopteran insect damage, comprising introducing lepidopteran insects infected with a virus comprising a genetically modified Heliothis virescens nudivirus (HvNV) having at least one mutation in a region selected from the group comprising HvNV Open Reading Frame 90 (HvNV ORF90) (SEQ ID NO: 21), HvNV Open Reading Frame 92 (HvNV ORF92) (SEQ ID NO: 22), and HvNV Persistence-associated Gene (HvNV pag1) (SEQ ID NO: 26), wherein said virus causes increased lepidopteran insect sterility in said lepidopteran insect introduced into said crop.

The crop may be selected from any one of corn, cotton, soybeans, tomatoes, sorghum, artichoke, asparagus, cabbage, cantaloupe, collard, cowpea, cucumber, eggplant, lettuce, lima bean, melon, okra, pea, pepper, potato, pumpkin, snap bean, spinach, squash, sweet potato, and watermelon, alfalfa, clover, cotton, flax, oat, millet, rice, sorghum, soybean, sugarcane, sunflower, tobacco, vetch, and wheat, avocado, grape, peaches, pear, plum, raspberry, strawberry, carnation, geranium, gladiolus, nasturtium, rose, snapdragon, zinnia, and combinations thereof.

The lepidopteran insect may be selected from Helicoverpa zea (H. zea) H. armigera, H. assulta, Heliothis virescens, Agrotis ipsilon, Spodoptera frugiperda, Spodoptera exiguae.

An implementation of the present invention may comprise a method of reducing a lepidopteran insect population, comprising introducing into said lepidopteran insect population a lepidopteran insect infected with a virus comprising a genetically modified Heliothis virescens nudivirus (HvNV) having at least one mutation in a region selected from the group comprising HvNV Open Reading Frame 90 (HvNV ORF90) (SEQ ID NO: 21), HvNV Open Reading Frame 92 (HvNV ORF92) (SEQ ID NO: 22), and HvNV Persistence-associated Gene (HvNV pag1) (SEQ ID NO: 26), wherein said-genetically modified Heliothis virescens nudivirus (HvNV) causes increased lepidopteran insect sterility in said lepidopteran insect population.

The lepidopteran insect is selected from Helicoverpa zea (H. zea) H. armigera, H. assulta, Heliothis virescens, Agrotis ipsilon, Spodoptera frugiperda, Spodoptera exiguae, and combinations thereof.

The lepidopteran insect population may comprise Bt resistant lepidopteran insects.

The genetically modified Heliothis virescens nudivirus (HvNV) may have a modification in one or more regions selected from the pag1 gene (SEQ ID NO: 26), the ORF 90 gene (SEQ ID NO: 21), or ORF 92 gene (SEQ ID NO: 22), wherein said modification disrupts or reduces expression from said region.

An implementation of the present invention may comprise a method of reducing a lepidopteran population comprising introducing into a lepidopteran insect population a virus comprising a genetically modified Heliothis virescens nudivirus (HvNV) having at least one mutation in a region selected from the group comprising HvNV Open Reading Frame 90 (HvNV ORF90) (SEQ ID NO: 21), HvNV Open Reading Frame 92 (HvNV ORF92) (SEQ ID NO: 22), and HvNV Persistence-associated Gene (HvNV pag 1) (SEQ ID NO: 26), wherein said virus is introduced by feeding the virus to larvae or adults, wherein said virus causes increased sterility in said lepidopteran population as compared to a wild-type HvNV virus.

An implementation of the present invention may comprise a method of preparing insect eggs for protecting a crop from lepidopteran insect damage, comprising: providing lepidopteran insect eggs that are infected with the active ingredient HzNV2 isolate 90DR71 or similar viruses with genes ORF 90, ORF 92 or PAG1 inactivated by genetic mutation, CRISPR modification or recombinant DNA methodology; disrupting a biological secretion from oviduct glands that adheres the egg to plant surfaces by minor physical force; and adding a powder or liquid formulation to the eggs so that the eggs can be subsequently dosed to a crop, thereby preparing the insect eggs for protecting a crop.

The lepidopteran insect eggs are selected from Helicoverpa zea (H. zea), H. armigera, H. assulta, Heliothis virescens, Agrotis ipsilon, Spodoptera frugiperda, Spodoptera exiguae, wherein the physical force is such as brushing the eggs off with a facial brush or by vigorous agitation such as a 1-minute shaking with a Vortex laboratory agitator, wherein the formulation is a powder formulation that comprises the eggs with an anti-clumping agents selected from at least one of cornstarch, flour, or vermiculite and a fluorescent dye, wherein the formulation is a liquid formulation that comprises the eggs with a carrier solution selected from at least one of a saline solution, water, or buffered solution, and an additive selected from at least one of a surfactant, suspending agent or wetting agent, and wherein the surfactant is selected from at least one of plant-safe dish soap, or polysorbate 20, the suspending agent is selected from at least one of a Xanthan gum, guar gum, or carboxymethyl cellulose (CMC) and the wetting agent is polyalkyleneoxide modified heptamethyltrisiloxane.

An implementation of the present invention may comprise a method of protecting a crop from lepidopteran insect damage, comprising providing lepidopteran insect eggs that are infected with the active ingredient HzNV2 isolate 90DR71 or similar viruses with genes ORF 90, ORF 92 or PAG1 inactivated by genetic mutation, CRISPR modification or recombinant DNA methodology that are in a powder or liquid formulation; monitoring environmental conditions and humidity, temperature, and wind conditions for viability and effective distribution of eggs; and applying the powdered or liquid formulated eggs at an application rate from 1,000 eggs per acre to 400,000 eggs per acre, wherein the powdered formulation is applied in the morning or the liquid formulation is applied in the afternoon or evening, wherein the environmental conditions are selected from at least one of wind, heat, and rain, wherein the application of the egg formulations is to a refuge plot, treating alternating rows within a field or applying infected eggs to every 10^{th} crop row, wherein said crop is selected from any one of corn, cotton, soybeans, tomatoes, sorghum, artichoke, asparagus, cabbage, cantaloupe, collard, cowpea, cucumber, eggplant, lettuce, lima bean, melon, okra, pea, pepper, potato, pumpkin, snap bean, spinach, squash, sweet potato, and watermelon, alfalfa, clover, cotton, flax, oat, millet, rice, sorghum, soybean, sugarcane, sunflower, tobacco, vetch, and wheat, avocado, grape, peaches, pear, plum, raspberry, strawberry, carnation, geranium, gladiolus, nasturtium, rose, snapdragon, zinnia, and combinations thereof, wherein said lepidopteran insect eggs are selected from Helicoverpa zea (H. zea), H. armigera, H. assulta, Heliothis virescens, Agrotis ipsilon, Spodoptera frugiperda, Spodoptera exiguae, wherein the lepidopteran insect egg population comprises Bt resistant lepidopteran insect eggs and wherein the genetically modified Heliothis virescens nudivirus (HvNV) has a modification in one or more regions selected from the pag1 gene (SEQ ID NO: 26), the ORF 90 gene (SEQ ID NO: 21), or ORF 92 gene (SEQ ID NO: 22), wherein said modification disrupts or reduces expression from said region.

An implementation of the present invention may comprise a lepidopteran insect infected with a virus comprising a genetically modified Heliothis virescens nudivirus (HvNV) having at least one mutation in a region selected from the group comprising HvNV Open Reading Frame 90 (HvNV ORF90) (SEQ ID NO: 21), HvNV Open Reading Frame 92 (HvNV ORF92) (SEQ ID NO: 22) and HvNV Persistence-associated Gene (HvNV pag1) (SEQ ID NO: 26) to disrupt expression of the gene product encoded by said mutant region; diapausing the virus-infected lepidopteran insect as a pupae from infected moths by: injecting 10 μL of a viral suspension that comprises 1×10⁶ viral particles in PBS at a pH of 6.8 into adult female moths that are 24 hours post-eclosion, wherein the injections are performed in the lower third of the female moth abdomen using standard microinjection techniques, placing injected females moths with uninfected males at a 1:1 ratio, allowing the females moths and uninfected males to mate under standard laboratory conditions at 30° C. with long-day photoperiod light to dark cycles of 14 hours light to 10 hours dark, discarding first 24 hour post-mating eggs, collecting eggs at 48 hours post-mating; preparing a polylactic acid (PLA) grid container with a light penetrating clear lid by adding corn earworm diet into the grids of the container, placing collected eggs into separate grids containing diet within the (PLA) grid container and securing the lid to the container after egg placement, rearing produced larvae under standard laboratory conditions at 30° C. with long-day photoperiod light to dark cycles of 14 hours light to 10 hours dark until larvae reach the third instar stage of development, transferring entire (PLA) grid container containing third instar stage larvae to an incubator or rearing room set at a temperature of 17.5° C. with a short-day photoperiod light to dark cycles of 8 hours light to 16 hours to dark induce diapause, rearing the larvae until they pupate, moving pupae to either an empty grid of the (PLA) grid container, or to individual empty cups, securing lids to the (PLA) grid container, or individual cups, confirming diapause at the pupal stage based on morphological characteristics of the continuing presence of larval eye spots on the pupal case, maintaining pupae in diapause for up to 2 months in a maintaining period, terminating diapause after the maintaining period ends by transferring the maintained pupae to a rearing room maintained under standard laboratory conditions at 30° C. with long-day photoperiod light to dark cycles of 14 hours light to 10 hours dark, which promotes continuation of development and adult emergence, and verifying upon emergence the infection status of the moths through visual morphology of agonadal status and/or viral plug presence in females, or molecular detection via polymerase chain reaction (PCR), wherein the lepidopteran insect is selected from Helicoverpa zea (H. zea), H. armigera, H. assulta, Heliothis virescens, Agrotis ipsilon, Spodoptera frugiperda, Spodoptera exiguae, and wherein said virus causes increased lepidopteran insect sterility as compared to a wild-type HvNV virus.

### REFERENCES

Adamo, S.A., Kovalko, I., Easy, R.H., and Stoltz, D. (2014). A viral aphrodisiac in the cricket Gryllus texensis. J Exp Biol. 217 (Pt 11):1970-1976, PMID: 24625650.
Ali, M.I., and Luttrell, R.G. (2007). Susceptibility of bollworm and tobacco budworm (Lepidoptera: Noctuidae) to Cry2Ab2 insecticidal protein. J Econ Entomol 100:921-931, PMID: 17598557.
Ali, M.I., Luttrell, R.G., and Young, S.Y. 3^{rd} (2006). Susceptibilities of Helicoverpa zea and Heliothis virescens (Lepidoptera: Noctuidae) populations to Cry1Ac insecticidal protein. J Econ Entomol 99:164-175, PMID: 16573337.
Arneodo, J.D., Balbi, E.I., Flores, F.M., and Sciocco-Cap, A. (2015). Molecular identification of Helicoverpa armigera (Lepidoptera: Noctuidae: Heliothinae) in Argentina and development of a novel PCR-RFLP method for its rapid differentiation from H. zea and H. gelotopoeon. J Econ Entomol 108(6): 2505-2510, PMID: 26318007.
Bedford, G.O. (2013). Biology and management of palm dynastid beetles: recent advances. Annu Rev Entomol. 58:353-372, PubMed PMID: 23317044.
Bergé, J.B., and Ricroch, A.E. (2010). Emergence of minor pests becoming major pests in GE cotton in China: what are the reasons? What are the alternatives practices to this change of status? GM Crops. 1(4):214-219, PMID: 21844676.

Bézier, A., Thézé, J., Gavory, F., Gaillard, J., Poulain, J., Drezen, J.M, and Herniou, E.A. (2015). The genome of the nucleopolyhedrosis-causing virus from Tipula oleracea sheds new light on the Nudiviridae family. J Virol. 89(6): 3008-3025, PMID: 25540386.
Burand, J.P. (2013). Pathology and replication of the sexually transmitted insect virus HzNV-2. Advances in Virus Research. ISBN: 978-1477555-04-0. iConcept Press.
Burand, J.P., Kim, W., Afonso, C.L., Tulman, E.R., Kutish, G.F., Lu, Z., and Rock, D.L. (2012). Analysis of the genome of the sexually transmitted insect virus Helicoverpa zea nudivirus 2. Viruses 4:28-61, PMID: 22355451.
Burand J.P., Tan, W., Kim, W., Nojima, S., and Roelofs, W. (2005). Infection with the insect virus Hz-2v alters mating behavior and pheromone production in female Helicoverpa zea moths. J Insect Sci. 5:6. PMID: 16299596.
Burand, J.P., and Rallis, C.P. (2004). In vivo dose-response of insects to Hz-2V infection. Virology journal 1:15, PMID: 15613241.
Burand, J.P., Rallis, C.P., and Tan, W. (2004). Horizontal transmission of Hz-2V by virus infected Helicoverpa zea moths. J Invertebr Pathol 85:128-131, PMID: 15050843.
Burand, J.P., and Lu, H. (1997). Replication of a Gonad-Specific Insect Virus in TN-368 Cells in Culture. J Invertebr Pathol. 70(2): 88-95. PMID: 9281395.
Burand, J.P., and Wood, H.A. (1986). Intracellular protein synthesis during standard and defective Hz-1 virus replication. J. Gen. Virol. 67:167-173.
Burand, J.P., Stiles, B., and Wood, H.A. (1983). Structural and Intracellular Proteins of the Nonoccluded Baculovirus HZ-1. J Virol. 46(1): 137-142. PMID: 16789238.
Burand, J.P., Wood, H.A., and Summer, M.D., (1983). Defective particles from a persistent baculovirus infection in Trichoplusia ni tissue culture cells. J. Gen. Virol. 64:391-398.
CABI 2015 HyperTextTransferProtocol://WorldWideWeb.cabi.org/isc/datasheet/26776, wherein “HyperTextTransferProtocol” is “http”, and “WorldWideWeb” is “www”
Campagne, P., Kruger, M., Pasquet, R., Le Ru, B., and Van den Berg, J. (2013). Dominant inheritance of field-evolved resistance to Bt corn in Busscolafusca. PLOS One 8: c69675, PMID: 23844262.
Capinera 2000, revised 2007 UF/IFAS Extension HyperTextTransferProtocol://entomology.ifas.ufl.edu/creatures, wherein “HyperTextTransferProtocol” is “http”
Carriere, Y., Crowder, D.W., and Tabashnik, B.E. (2010). Evolutionary ecology of insect adaptation to Bt crops. Evol Appl 3:561-573, EPA (1998). The Environmental Protection Agency's White Paper on Bt Plant-pesticide Resistance Managment (EPA).
Chao, Y.C., Lee, S.T., Chang, M.C., Chen, H.H., Chen, S.S., Wu, T.Y., Liu, F.H., Hsu, E.L., and Hou, R.F. (1998). A 2.9-kilobase noncoding nuclear RNA functions in the establishment of persistent Hz-1 viral infection. J Virol. 72(3):2233-2245. PMID: 9499081.
Chao, Y.C., Wood, H.A., Chang, C.Y., Lee, H.J., Shen, W.C., and Lee, H.T. (1992). Differential expression of Hz-1 baculovirus genes during productive and persistent viral infections. J Virol. 66(3): 1442-7448. PMID: 1738201.
Chen, H.H., Tsai, F.Y., Chen, C.T. (2001). Negative regulatory regions of the PAT1 promoter of Hz-1 virus contain GATA elements which associate with cellular factors and regulate promoter activity. J Gen Virol. 82(Pt 2): 313-320. PMID: 11161268.
Cheng, C.H., Liu, S.M., Chow, T.Y., Hsiao, Y.Y., Wang, D.P., Huang, J.J., Chen, H.H. (2002). Analysis of the complete genome sequence of the Hz-1 virus suggests that it is related to members of the Baculoviridae. J Virol. 76(18): 9024-9034. PMID: 12186886.
Cheng, R.L., Xi, Y., Lou, Y.H., Wang, Z., Xu, J.Y., Xu, H.J., and Zhang, C.X. (2014). Brown planthopper nudivirus DNA integrated in its host genome. J Virol. 88(10): 5310-5318. PMID: 24574410.
Cunningham, J.P., and Zalucki, M.P. (2014). Understanding heliothine (Lepidoptera: Heliothinae) pests: what is a host plant? J Econ Entomol. 107(3): 881-896, PMID: 25026644.
de Miranda, J.R., and Fries, I. (2008). Venereal and vertical transmission of deformed wing virus in honeybees (Apis mellifera L.). J Invertebr Pathol. 98(2): 184-189, PubMed PMID: 18358488.
Ferrelli, M.L., Taibo, C., Fichetti, P., Sciocco-Cap, A., Arneodo, J.D. (2015). Characterization of a new Helicoverpa armigera nucleopolyhedrovirus variant causing epizootic on a previously unreported host, Helicoverpa gelotopoeon (Lepidoptera: Noctuidae). J Invertebr Pathol. pii: S0022-2011(15)30008-2, PMID: 26296927.
Fitt, G.P. (1989). The ecology of Heliothis species in relation to agrosystems. Ann Rev Entomol 34, 17-52
Franz, G., and Robinson, A.S. (2011). Molecular technologies to improve the effectiveness of the sterile insect technique. Genetica 139(1): 1-5, PMID: 21258957.
Gherezghiher, T.B., Ming, X., Villalta, P.W., Campbell, C., and Tretyakova, N.Y. (2013). 1,2,3,4-Diepoxybutane-induced DNA-protein cross-linking in human fibrosarcoma (HT1080) cells. J Proteome Res. 12(5): 2151-2164, PMID: 23506368.
Granados, R. R., Nguyen, T., and Cato, B. (1978). An insect cell line persistently infected with a baculovirus-like particle. Intervirol, 10(5), 309-17.
Hamm, J.J. (1997). Gonad-specific virus of Helicoverpa zea does not affect infectivity of nuclear polyhedrosis virus. J. Entomol. Sci. 32(1): 106-109
Hamm, J.J., Carpenter, J.E., and Styer, E.L. (1996). Oviposition day effect on incidence of agonadal progeny of Helicoverpa zea (Lepidoptera: Nocutidae) infected with a virus. Ann. Entomol. Soc. Am. 89(2): 266-275
Helicoverpa armigera TWG Report, Rev. 1, USDA APHIS PPQ Technical Working Group
HyperTextTransferProtocol://WorldWideWeb.statista.com/topics/2062/genetically-modified-crops/, wherein “HyperTextTransferProtocol” is “http,” and “WorldWideWeb” is “www”

Huang, F., Andow, D.A., and Buschman, L. (2011). Success of the high dose/refuge resistance management strategy after 15 years of Bt crop use in North America. Entomol Exp Appl 140, 1-16.
James, C. (2012). Global status of commercialized biotech/GM crops: 2012. In ISAA Briefs (Ithaca, NY: ISAAA).
Kempf, C., Michel, M.R., Omar, A., Jentsch, P., and Morell, A. (1990). Semliki Forest virus induced cell-cell fusion at neutral extracellular pH. Biosci Rep. 10(4): 363-374, PMID: 2249002.
Knell, R.J., and Webberley, K.M. (2004). Sexually transmitted diseases of insects: distribution, evolution, ecology and host behaviour. Biol Rev Camb Philos Soc. 79(3): 557-581, PMID: 15366763.
Lee, S., Park, K.H., Nam, S.H., Kwak, K.W., and Choi, J.Y. (2015). First report of Oryctes rhinoceros nudivirus (Coleoptera: Scarabacidae) causing severe disease in Allomyrina dichotoma in Korea. J Insect Sci. 12:15, PubMed PMID: 25765317.
Lee, J.C., Chen, H.H., Wei, H.L., and Chao, Y.C. (1993). Superinfection-induced apoptosis and its correlation with the reduction of viral progeny in cells persistently infected with Hz-1 baculovirus. J Virol. 67(12): 6989-94, PMID: 8230422.
Lin, C.L., Lee, J.C., Chen, S.S., Wood, H.A., Li, M.L., Li, C.F., and Chao, Y.C. (1999). Persistent Hz-1 virus infection in insect cells: evidence for insertion of viral DNA into host chromosomes and viral infection in a latent status. J Virol. 73(1): 128-39, PMID: 9847315.
Lupiani, B., Raina, A.K., and Huber, C. (1999). Development and use of a PCR assay for detection of the reproductive virus in wild populations of Helicoverpa zea (Lepidoptera: noctuidae). J Invertebr Pathol 73, 107-112.
Luttrell, R.G., and Jackson, R.E. (2012). Helicoverpa zea and Bt cotton in the United States. GM Crops & Food: Biotechnol in Agriculture Food Chain, 3(3): 213-227.
Morrison, N.I., Simmons, G.S., Fu, G., O'Connell, S., Walker, A.S., Dafa'alla, T., Walters, M., Claus, J., Tang, G., Jin, L., Marubbi, T., Epton, M.J., Harris, C.L., Staten, R.T., Miller, E., Miller, T.A., and Alphey, L. (2012). Engineered repressible lethality for controlling the pinkbollworm, a lepidopteran pest of cotton. PLoS One 7(12): e50922, PMID: 23226548.
Nguyen, Q., Chan, L.C., Nielsen, L.K., and Reid, S. (2013). Genome scale analysis of differential mRNA expression of Helicoverpa zea insect cells infected with a H. armigera baculovirus. Virology 444(1-2): 158-170, PMID: 23827436.
Nguyen, Q., Palfreyman, R.W., Chan, L.C., Reid, S., and Nielsen, L.K. (2012). Transcriptome sequencing of and microarray development for a Helicoverpa zea cell line to investigate in vitro insect cell-baculovirus interactions. PLoS One. 7(5): e36324, PMID: 22629315.
U.S. Pat. No. 7,476,499: “Methods of identifying anti-viral agents” (use of DEB as mutagen)
U.S. Pat. No. 7,261,886 B2 “Insect larva aerosol infection method for producing recombinant proteins and baculovirus bio-insecticides”
patent #US 2011/0314562 A1 “Insect infection method for production of proteins”
J. R. Phillips, L. D. Newsom, Diapause in Heliothis zea and Heliothis virescens (Lepidoptera: Noctuidae), Annals of the Entomological Society of America, Volume 59, Issue 1, 1966, Pages 154-159, https://doi.org/10.1093/aesa/59.1.154, wherein “HyperTextTransferProtocol” is “http.”
Pichon, A., Bézier, A., Urbach, S., Aury, J.M., Jouan, V., Ravallec, M., Guy, J., Cousserans, F., Thézé, J., Gauthier, J., Demettre, E., Schmieder, S., Wurmser, F., Sibut, V., Poirié, M., Colinet, D., da Silva, C., Couloux, A., Barbe, V., Drezen, J.M., and Volkoff, A.N. (2015). Recurrent DNA virus domestication leading to different parasite virulence strategies. Sci Adv. 1(10): e1501150, PMID: 26702449.
Pushparajan, C., Claus, J.D., Marshall, S.D., and Visnovsky, G. (2013). Characterization of growth and Oryctes rhinoceros nudivirus production in attached cultures of the DSIR-HA-1179 coleopteran insect cell line. Cytotechnology. 65(6): 1003-1016, PMID: 23979321.
Raina, A.K., Adams, J.R., Lupiani, B., Lynn, D.E., Kim, W., Burand, J.P., and Dougherty, E.M. (2000). Further characterization of the gonad-specific virus of corn earworm, Helicoverpa zea. J Invertebr Pathol 76:6-12, PMID: 10963397.
Raina, A.K., and Lupiani, B. (2006). Acquisition, persistence, and species susceptibility of the Hz-2V virus. J Invertebr Pathol 93:71-74.
Rallis, C.P., and Burand, J.P. (2002a). Pathology and ultrastructure of Hz-2V infection in the agonadal female corn earworm, Helicoverpa zea. J Invertebr Pathol 81:33-44, PMID: 12417211.
Rallis, C.P., and Burand, J.P. (2002b). Pathology and ultrastructure of the insect virus, Hz-2V, infecting agonadal male corn earworms, Helicoverpa zea. J Invertebr Pathol 80:81-89, PMID: 12383433.
Reardon, J.T., Liljestrand-Golden, C.A., Dusenbery, R.L., and Smith, P.D. (1987). Molecular Analysis of Diepoxybutane-Induced Mutations at the rosy Locus of Drosophila melanogaster. Genetics 115:323-331, PMID: 17246369.
Simmons, G.S., McKemey, A.R., Morrison, N.I., O′Connell, S., Tabashnik, B.E., Claus, J., Fu, G., Tang, G., Sledge, M., Walker, A.S., Phillips, C.E., Miller, E.D., Rose, R.I., Staten, R.T., Donnelly, C.A., and Alphey, L. (2011). Field performance of a genetically engineered strain of pink bollworm. PLOS One. 6(9): e24110, PMID: 21931649.
Skalsky, R.L., and Cullen, B.R. (2010). Viruses, microRNAs, and host interactions. Annu Rev Microbiol. 64:123-41, PMID: 20477536.
Smith-Pardo, A. (2014). The old world bollworm Helicoverpa armigera (Hübner) (Lepidoptera: Noctuidae: Heliothinae) its biology, economic importance and its recent introduction into the western hemisphere. Boletin del museo entomológico 6(1): 18-28.
Tabashnik, B.E., Mota-Sanchez, D., Whalon, M.E., Hollingworth, R.M., and Carrière, Y. (2014). Defining terms for proactive management of resistance to Bt crops and pesticides. J Econ Entomol 107(2): 496-507, PMID: 24772527.
Tabashnik, B.E., Brevault, T., and Carriere, Y. (2013). Insect resistance to Bt crops: lessons from the first billion acres. Nat Biotechnol 31:510-521, PMID: 23752438.
Tabashnik, B.E., Van Rensburg, J.B.J., and Carriere, Y. (2009). Field-evolved insect resistance to Bt crops: evidence versus theory. J Econ Entomol 102:2011-2025.
Tretyakova, N.Y., Michaelson-Richie, E.D., Gherezghiher, T.B., Kurtz, J., Ming, X., Wickramaratne, S., Campion, M., Kanugula, S., Pegg, A.E., and Campbell, C. (2013). DNA-reactive protein monoepoxides induce cell death and mutagenesis in mammalian cells. Biochemistry 52(18): 3171-3181, PMID: 23566219.
Tyagi, A., Singh, N.K., Gurtler, V., and Karunasagar, I. (2016). Bioinformatics analysis of codon usage patterns and influencing factors in Penaeus monodon nudivirus. Arch Virol. 161(2): 459-464, PMID: 26586333.
Unckless, R.L. (2011). A DNA virus of Drosophila. PLoS One. 6(10): e26564, PMID: 22053195.
Vargas, Roger, and Toshiyuki Nishida. “Life table of the corn earworm, Heliothis zea (Boddie), in sweet corn in Hawaii.” (1980).
Walters, M., Morrison, N.I., Claus, J., Tang, G., Phillips, C.E., Young, R., Zink, R.T., and Alphey, L. (2012). Field longevity of a fluorescent protein marker in an engineered strain of the pink bollworm, Pectinophora gossypiella (Saunders). PLoS One 7(6): e38547, PMID: 22693645.
Wang, Y., Bininda-Emonds, O.R., van Oers, M.M., Vlak, J.M., and Jehle, J.A. (2011). The genome of Oryctes rhinoceros nudivirus provides novel insight into the evolution of nucleararthropod-specific large circular double-stranded DNA viruses. Virus Genes 42(3): 444-456, PMID: 21380757.
Wang, Y., and Jehle, J.A. (2009). Nudiviruses and other large, double-stranded circular DNA viruses of invertebrates: new insights on an old topic. J Invertebr Pathol. 101(3): 187-193, PMID: 19460388.
Wang, Y., Kleespies, R.G., Ramle, M.B., and Jehle, J.A. (2008). Sequencing of the large dsDNA genome of Oryctes rhinoceros nudivirus using multiple displacement amplification of nanogram amounts of virus DNA. J Virol Methods. 152(1-2): 106-108, PMID: 18598718.
Wang, Y., Kleespies, R.G., Huger, A.M., and Jehle, J.A. (2007). The genome of Gryllus bimaculatus nudivirus indicates an ancient diversification of baculovirus-related nonoccluded nudiviruses of insects. J Virol. 81(10): 5395-5406, PMID: 17360757.
Wang, Y., van Oers, M.M., Crawford, A.M., Vlak, J.M., and Jehle, J.A. (2007). Genomic analysis of Oryctes rhinoceros virus reveals genetic relatedness to Heliothis zea virus 1. Arch Virol. 152(3): 519-531, PMID: 17106621.
Stanley G. Wellso, Perry L. Adkisson, A long-day short-day effect in the photoperiodic control of the pupal diapause of the bollworm, Heliothis zea (Boddie), Journal of Insect Physiology, Volume 12, Issue 11, 1966, Pages 1455-1465, ISSN 0022-1910, https://doi.org/10.1016/0022-1910(66)90160-0, wherein “HyperTextTransferProtocol” is “http.”
Wijen, J.P., Nivard, M.J., and Vogel, E.W. (2001). Genetic damage by bifunctional agents in repair-active pre-meiotic stages of Drosophila males. Mutat Res 478:107-117, PMID: 11406175.
Wood, H.A., and Burand, J.P. (1986). Persistent and productive infections with the Hz-1 baculovirus. Current topics in microbiology and immunology 131:119-133, PMID: 3816297.
Wu, Y.L., Wu, C.P., Lee, S.T., Tang, H., Chang, C.H., Chen, H.H., and Chao, Y.C. (2010). The early gene hhil reactivates Heliothis zea nudivirus 1 in latently infected cells. Journal of virology 84:1057-1065, PMID: 19889784.
Wu, Y.L., Wu, C.P., Liu, C.Y., Hsu, P.W., Wu, E.C., and Chao, Y.C. (2011a). A non-coding RNA of insect HzNV-1 virus establishes latent viral infection through microRNA. Scientific reports 1:60, PMID: 22355579.
Wu, Y.L., Wu, C.P., Liu, C.Y., Lee, S.T., Lec, H.P., and Chao, Y.C. (2011b). Heliothis zea nudivirus 1 gene hhil induces apoptosis which is blocked by the Hz-iap2 gene and a noncoding gene, pag1. Journal of virology 85:6856-6866, PMID: 21543471.
Wu, Y.L., Liu, C.Y., Wu, C.P., Wang, C.H., Lee, S.T., and Chao, Y.C. (2008). Cooperation of ie1 and p35 genes in the activation of baculovirus AcMNPV and HzNV-1 promoters. Virus Res. 135(2): 247-254, PMID: 18486255.
WorldWide Web.usda.gov/nass/PUBS/TODAYRPT/acrg0615.pdf (USDA “Acreage” 2015 ISSN: 1949-1522), wherein “WorldWideWeb” is “www”
Yang, Y.T., Lec, D.Y., Wang, Y., Hu, J.M., Li, W.H., Leu, J.H., Chang, G.D., Ke, H.M., Kang, S.T., Lin, S.S., Kou, G.H., and Lo, C.F. (2014). The genome and occlusion bodies of marine Penaeus monodon nudivirus (PmNV, also known as MBV and PemoNPV) suggest that it should be assigned to a new nudivirus genus that is distinct from the terrestrial nudiviruses. BMC Genomics 15:628, PMID: 25063321.
Yazaki, K., Mizuno, A., Sano, T., Fujii, H., and Miura, K. (1986). A new method for extracting circular and supercoiled genome segments from cytoplasmic polyhedrosis virus. J Virol Methods 14 (3-4): 275-283, PMID: 3539959.
Zhao, X.-C., Dong, J.-F., Tang, Q.-B., Yan, Y.-H., Gelbic, I., Van Loon, J.J.A., and Wang, C.-Z. (2005). Hybridization between Helicoverpa armigera and Helicoverpa assulta (Lepidoptera: Noctuidae): development and morphological characterization of F₁ hybrids. Bulletin of Entomol Research 95:409-416.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A lepidopteran insect infected with a virus comprising a genetically modified Heliothis virescens nudivirus (HvNV) having at least one mutation in a region selected from the group comprising HvNV Open Reading Frame 90 (HvNV ORF90) (SEQ ID NO: 21), HvNV Open Reading Frame 92 (HvNV ORF92) (SEQ ID NO: 22) and HvNV Persistence-associated Gene (HvNV pag1) (SEQ ID NO: 26) to disrupt expression of the gene product encoded by said mutant region, wherein said virus causes increased lepidopteran insect sterility as compared to a wild-type HvNV virus.

2. The insect of claim 1, wherein said lepidopteran insect is selected from the group consisting of Helicoverpa zea (H. zea), H. armigera, H. assulta, Heliothis virescens, Agrotis ipsilon, Spodoptera frugiperda, and Spodoptera exiguae.

3. A method of making a lepidopteran insect capable of transmitting a virus comprising infecting a lepidopteran insect with a genetically modified Heliothis virescens nudivirus (HvNV) having at least one mutation in a region selected from the group comprising HvNV Open Reading Frame 90 (HvNV ORF90) (SEQ ID NO:21), HvNV Open Reading Frame 92 (HvNV ORF92) (SEQ ID NO: 22), and HvNV Persistance-associated Gene (HvNV pag1) (HvNV ORF92) (SEQ ID NO: 26), to disrupt expression of the gene product encoded by said mutant region, wherein said virus causes increased lepidopteran insect sterility in said lepidopteran insect, thereby infecting said lepidopteran insect with said virus.

4. The method of claim 3, wherein said lepidopteran insect is selected from Helicoverpa zea (H. zea), H. armigera, H. assulta, Heliothis virescens, Agrotis ipsilon, Spodoptera frugiperda, Spodoptera exiguae.

5. The method of claim 3, wherein said virus is derived from a viral plug.

6. The method of claim 3, wherein said virus is administered orally to said lepidopteran insect.

7. The method of claim 3, wherein said virus is administered to said lepidopteran insect via direct inoculation of insect larvae or adult lepidopteran insect by puncturing the cuticle with a pin containing viral inoculum derived from a viral plug.

8. The method of claim 3, wherein said virus is administered to said insect via direct hypodermic injection into third instar larvae or moths.

9. A method of preparing insect eggs for protecting a crop from lepidopteran insect damage, comprising:
providing lepidopteran insect eggs that are infected with the active ingredient HzNV2 isolate 90DR71 or similar viruses with genes ORF 90, ORF 92 or PAG1 inactivated by genetic mutation, CRISPR modification or recombinant DNA methodology;
disrupting a biological secretion from oviduct glands that adheres the egg to plant surfaces by minor physical force; and
adding a powder or liquid formulation to the eggs so that the eggs can be subsequently dosed to a crop, thereby preparing the insect eggs for protecting a crop.

10. The method of claim 9, wherein said lepidopteran insect eggs are selected from Helicoverpa zea (H. zea), H. armigera, H. assulta, Heliothis virescens, Agrotis ipsilon, Spodoptera frugiperda, Spodoptera exiguae.

11. The method of claim 9, wherein the physical force is such as brushing the eggs off with a facial brush or by vigorous agitation such as a 1-minute shaking with a Vortex laboratory agitator.

12. The method of claim 9, wherein the formulation is a powder formulation that comprises the eggs with an anti-clumping agents selected from at least one of cornstarch, flour, or vermiculite and a fluorescent dye.

13. The method of claim 9, wherein the formulation is a liquid formulation that comprises the eggs with a carrier solution selected from at least one of a saline solution, water, or buffered solution, and an additive selected from at least one of a surfactant, suspending agent or wetting agent,
wherein the surfactant is selected from at least one of plant-safe dish soap, or polysorbate 20, the suspending agent is selected from at least one of a Xanthan gum, guar gum, or carboxymethyl cellulose (CMC) and the wetting agent is polyalkyleneoxide modified heptamethyltrisiloxane.
